(19) United States
(12) Patent Application Publication
(10) Pub. No.: US 2005/0267056 A1
(43) Pub. Date: Dec. 1, 2005

(54) Synthesis and manufacture of pentostatin and its precursors, analogs and derivatives

(76) Inventors: Pasit Phiasivongsa, Brentwood, CA (US); Sanjeev Redkar, Hayward, CA (US)

Correspondence Address: WILSON SONSINI GOODRICH & ROSATI, 650 PAGE MILL ROAD, PALO ALTO, CA, 94304-1050, US

(21) Appl. No.: 10/942,319

(22) Filed: Sep. 15, 2004

Related U.S. Application Data
(60) Provisional application No. 60/503,237, filed on Sep. 15, 2003.

(51) Int. Cl. A61K 31/7056; C07H 19/22

(52) U.S. Cl. 514/43; 536/27.1

(57) ABSTRACT

Methods and compositions are provided for efficiently preparing and manufacturing pentostatin. Also provided are novel precursors of pentostatin, pentostatin analogs and derivatives. In one aspect of the invention, a method is provided for total chemical synthesis of pentostatin via a route of heterocyclic ring expansion. For example, a heterocyclic pharmaceutical intermediate for drugs such as pentostatin, e.g., the diazepinone precursor, can be obtained efficiently through a ring expansion of an O—C—N functionality in a hypoxanthine or 2′-deoxyinosine derivative. The methods and compositions can also be used to synthesize and manufacture heterocyclic compounds other than pentostatin, especially pharmaceutically important heterocyclic compounds.

### CROSS REFERENCE TO RELATED APPLICATION

This application claims the benefit of U.S. Provisional Application No. 60/503,237, filed Sep. 15, 2003, which is incorporated herein by reference in its entirety.

### BACKGROUND OF THE INVENTION

1. Field of the Invention

The present invention relates to compositions and methods for preparing and manufacturing pentostatin ((8R)-3-(2-deoxy-β-D-erythro-pentofuranosyl)-3,6,7,8-tetrahydroimidazo[4,5-d][1,3]diazepin-8-ol), precursors of pentostatin, pentostatin analogs and derivatives, and other heterocycles that require expansion of the heterocyclic ring at an O—C—N functionality.

2. Description of Related Art

Pentostatin, (8R)-3-(2-deoxy-β-d-erythro-pentofuranosyl)-3,6,7,8-tetrahydroimidazo[4,5-d][1,3]diazepin-8-ol, is a potent the potent inhibitor of adenosine deaminase. The chemical structure of pentostatin is shown below:

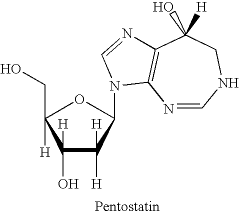

The total synthesis of pentostatin poses a challenge since the molecule contains (1) a unique and unstable heterocyclic base, (2) a 2-deoxy sugar that defies attempts at stereocontrolled glycosylation to favor the β-anomer, and (3) a central chiral hydroxyl group. The merit of any chemical transformation is measured by its resolutions to these three key difficulties.

The first synthesis of Pentostatin was demonstrated by Showalter and Baker of Warner-Lambert/Parke-Davis Pharmaceutical. Chan, E.; Putt, S. R.; Showalter, H. D. H.; Baker, D. C. J. Org. Chem. 1982, 47, 3457-3464. The procedure focused mainly on the synthesis of the heterocyclic base (FIGS. 1A-C). The crux of the procedure is synthesis of diamine precursor such as 7, which can have a ketone function (as shown) or a chiral alcohol group in the position a to the imidazole. The latter proposal has yet to be realized. Certain aspects of this approach make it flexible and amenable to improvement. Nevertheless, the procedure requires no less than 8 steps to synthesize just the base precursor 8a. The chemical structure of the base precursor 8a is shown below. Such a procedure is difficult to commercially reduce to practice and expensive to scale-up to manufacturing size. Column chromatography is required for purification at many of these steps.

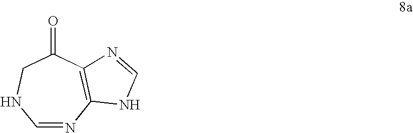

To further improve efficiency and minimize cost for manufacturing synthetic pentostatin, Chen et al. synthesized precursor 4 from a different starting material (FIG. 2). Chen, B.-C.; Chao, S. T.; Sundeen, J. E.; Tellew, J.; Ahmad, S. Tetrahedron Lett. 2002, 43, 1595-1596. The modifications eliminated the N-2 benzylation side-reaction (no formation of 3b), improved total yield of precursor 7 from 19% to 30%, and used less expensive starting material 1b.

Of the numerous methods available for glycosylation, once precursor 8a has been synthesized and purified, Showalter and Baker condensed it to the 2-deoxy sugar via a peracylglycosyl chloride adapted from the stannic chloride catalyzed process of Vorbrüggen (FIG. 1B) to generate two anomers of pentostatin precursors 9a and 9b. The chemical structures of the pentostatin precursors 9a and 9b are shown below. 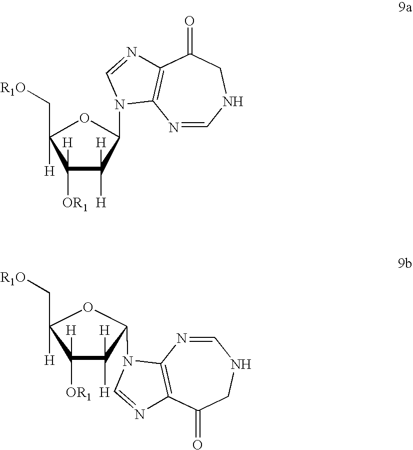
Chan, E.; Putt, S. R.; Showalter, H. D. H.; Baker, D. C. J. Org. Chem. 1982, 47, 3457-3464. There was no stereocontrol of the glycosylation, but the 1:1 anomeric mixture could be separated by traditional column chromatography or fractional crystallization. In addition to the multi-steps route shown in FIG. 1A, the peracylglycosyl chloride starting material must also be prepared by a multi-step procedure, which as a whole added to an already lengthy procedure that required isolation and purification steps.

After 9a (23%) had been isolated in pure form the protective group was removed and subsequently reduced with sodium borohydride to pentostatin (FIG. 1C, 10a), which converted the carbonyl functionality into a chiral hydroxyl group. However, since the transformation transpired without stereocontrol, a diastereomeric mixture of compounds 10a and 10b was obtained. The chemical structures of compounds 10a and 10b are shown below.

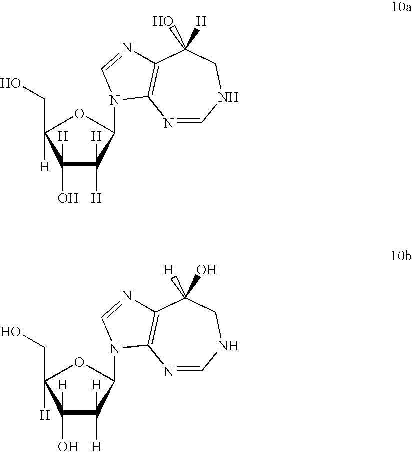

Various sterically hindered borohydrides (potassium tri-sec-butylborohydride and 9-borabicyclo[3.3.1]nonane; lithium tri-tert-butoxyaluminum hydride, lithium aluminum hydride-(−)-menthol complex, lithium aluminum hydride-(−)-N-methylephedrine-3,5-xylenol complex) were considered, but they found little improvement in enantio-selectivity or yield. Chan, E.; Putt, S. R.; Showalter, H. D. H.; Baker, D. C. J. Org. Chem. 1982, 47, 3457-3464. Separation of the 1:1 mixture of diastereomers 10a (33%) and 10b (29%) were determined best by a C-18 reverse-phase preparative HPLC for small scales. For larger scales, fractional crystallization was better.

Another approach to resolving the three key difficulties was proposed by Rapoport (Ho, J. Z.; Mohareb, R. M.; Ahn, J. H.; Sim, T. B.; Rapoport, H. J. Org Chem. 2003, 68, 109-114). This approach involved enantiocontrolled synthesis of the base with the natural R configuration of the hydroxyl group in place. To illustrate this approach, analogues of pentostatin were synthesized (FIG. 3; cyclopentyl analogue). First precursor 11 was obtained by a multi-steps procedure starting with L-methionine, which required at least 8 synthetic steps. Truong, T. V.; Rapoport, H. J. Org. Chem. 1993, 58, 6090-6096. However, synthesis of pentostatin itself has yet to be realized, which not only would have a lengthier process but also unresolved stereo-chemical difficulties with the sugar moiety.

The approach proposed by Rapoport is very promising. It resolves a key stereochemical difficulty by incorporating a carefully designed synthetic pathway. The one serious drawback is that it still involves several synthetic steps. Nevertheless, it more than matches the synthetic route proposed by Showalter and Baker.

Nature, in contrast, has a very efficient pathway to synthesize pentostatin. Hanvey et al. has identified 8-ketocoformycin and 8-ketodeoxycoformycin 9a as intermediates in the biosynthesis of coformycin and pentostatin by S. antibioticus (FIG. 4). Hanvey, J. C.; Hawkins, E. S; Tunac, J. B.; Dechter, J. J.; Baker, D. C.; Suhadolnik, R. J. Biochemistry 1987, 26, 5636-5641; and Hanvey, J. C.; Hawkins, E. S.; Baker, D. C.; Suhadolnik, R. J. Biochemistry 1988, 27, 5790-5795. Formation of the 1,3-diazepine ring comes about by a ring expansion of the adenine moiety of adenosine with the C-1 of D-ribose. Then, reduction of the 8-keto functional group occurs stereospecificly to either coformycin or pentostatin.

In view of the disadvantages associated with the different synthesis schemes of pentostatin described above, there exists a need for a high yield, efficient chemical synthesis of pentostatin, pentostatin derivatives and analogs, which does not require biosynthesis of pentostatin by microorganisms.

### SUMMARY OF THE INVENTION

Methods and compositions are provided for efficiently preparing and manufacturing pentostatin, its precursors, analogs and derivatives, and other heterocyclic compounds.

In one aspect of the invention, a method is provided for total chemical synthesis of pentostatin via a route of heterocyclic ring expansion. In one embodiment, the method comprises:
providing a hypoxanthine derivative wherein at least one of the imidazole secondary amine and the cyclic O—C—N functionality (i.e., O═C—NH or HO—C═N) is protected by a protective group;
expanding the 6-member ring of the hypoxanthine derivative to form a protected diazepinone precursor having the formula 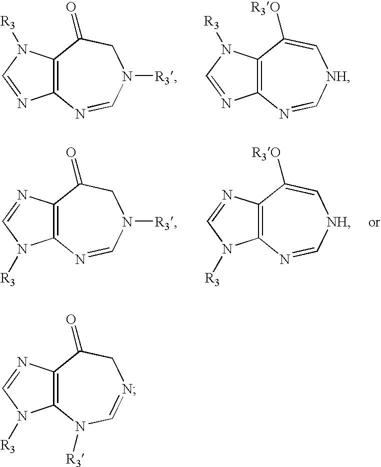
deprotecting the protected diazepinone precursor to yield a diazepinone precursor 8a having the formula 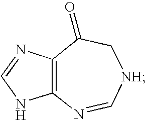
condensing the N-2 of the diazepinone precursor 8a with the C-1 of 2-deoxy-D-ribose or its derivative to yield an intermediate having the formula 9a 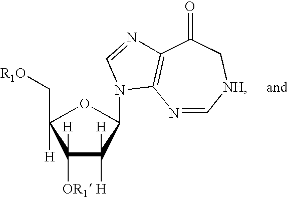
reducing the 8-keto functional group of compound 9a to yield pentostatin, wherein R₁ and R₁′ are each independently H or a protective group, and R₃ and R₃′ are each independently H or a protective group.

Examples of protective groups R₃ and R₃′ for the imidazole secondary amine and the cyclic O—C—N functionality include, but are not limited to: carbamates (e.g., methyl, ethyl, t-butyl, benzyl, 9-fluorenylmethyl, 2,2,2-trichloroethyl, 1-methyl-1-(4-biphenyl)ethyl, and 1-(3,5-di-t-butyl)-1-methylethyl); amides (e.g., acetamide, trifluoroacetamide, and benzamide); aryl amines (e.g., benzylamine, 4-methoxybenzylamine, and 2-hydroxybenzylamine); and silyl amines.

In another embodiment, the method comprises:
providing a 2′-deoxyinosine derivative wherein at least one of the hypoxanthine oxygen, the hypoxanthine amide nitrogen, the 3′-hydroxyloxygen, and 5′-hydroxyloxygen is protected by a protective group;
expanding the O—C—N functionality of the 6-member ring of a2′-deoxyinosine derivative to produce an intermediate having the formula 20a or 20b 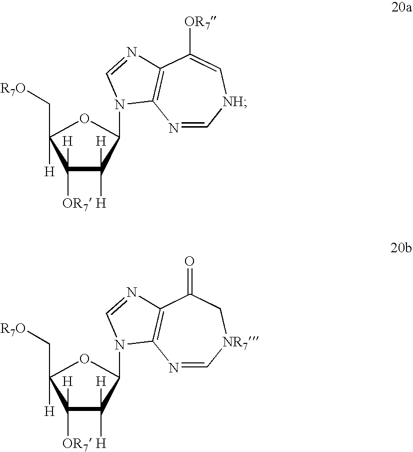
and
deprotecting and reducing the 8-keto functionality of compound 20a or 20b to yield pentostatin, wherein R₇, R₇′, R₇″ and R₇′″ are each independently H or a protective group.

Examples of protective groups R₇ and R₇′ include, but are not limited to benzyl ethers (e.g., p-methoxybenzyl, 3,4-dimethoxybenzyl, nitrobenzyl, and p-cyanobenzyl); silyl ethers (e.g., triakylsilyl and alkoxydialkylsilyl); esters (e.g., acetate, halogenatedacetate, alkoxyacetate, and benzoate).

Examples of protective group R₇″ include, but are not limited to, benzyl ethers (e.g., p-methoxybenzyl, 3,4-dimethoxybenzyl, nitrobenzyl, and p-cyanobenzyl); and silyl ethers (e.g., triakylsilyl and alkoxydialkylsilyl).

R₇′″ may be a carbamate protective group (e.g., methyl carbamate, ethyl carbamate, t-butyl carbamate, benzyl carbamate, 9-fluorenylmethyl carbamate, 2,2,2-trichloroethyl carbamate, 1-methyl-1-(4-biphenyl)ethyl carbamate, and 1-(3,5-di-t-butyl)-1-methylethyl carbamate).

According to the method, the O—C—N functionality of the 6-member ring of the 2′-deoxyinosine derivative may be expanded by reacting the 2′-deoxyinosine derivative with diazomethane or trimethylsilyldiazomethane in the presence of a Lewis acid catalyst, and preferably with anhydrous solution of diazomethane or trimethylsilyldiazomethane in ether.

Examples of the Lewis acid catalyst is include, but are not limited to, trimethylsilyl triflate (TMSOTf), BX₃, AlX₃, FeX₃, GaX₃, SbX₅, SnX₄, AsX₅, ZnX₂, and HgX₂, where X is a halogen. Preferably, the Lewis acid catalyst is ZnCl₂ or HgBr₂.

In another aspect of the invention, a method is provided for preparing coformycin. The method comprises
providing an inosine derivative wherein at least one of the hypoxanthine oxygen, the hypoxanthine amide nitrogen, the 2′-hydroxyloxygen, the 3′-hydroxyloxygen, and 5′-hydroxyl oxygen is protected by a protective group;
expanding the O—C—N functionality of the 6-member ring of the 2′-inosine derivative to produce an intermediate having the formula 21a or 21b 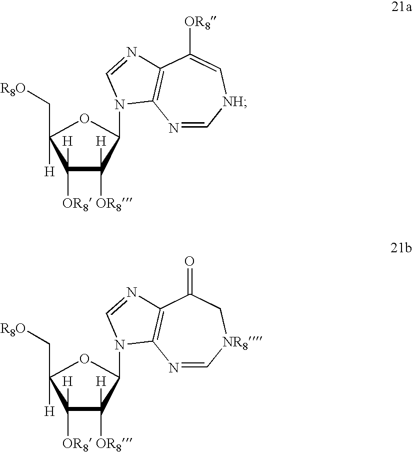
and
deprotecting and reducing the 8-keto functionality of compound 21a or 21b to yield coformycin, wherein R₈, R₈′, R₈″, R₈′″ and R₈″″ are each independently H or a protective group.

The R₈, R₈′ and R₈′″ protective groups may be benzyl ethers, silyl ethers, or esters. R₈″ protective group may be a benzyl ether and silyl ether. R₈″″ may be a carbamate protective group.

According to the method, the O—C—N functionality of the 6-member ring of the inosine derivative may be expanded by reacting the inosine derivative with diazomethane or trimethylsilyldiazomethane in the presence of a Lewis acid catalyst, and preferably with anhydrous solution of diazomethane or trimethylsilyldiazomethane in ether.

Examples of the Lewis acid catalyst is include, but are not limited to, trimethylsilyl triflate (TMSOTf), BX₃, AlX₃, FeX₃, GaX₃, SbX₅, SnX₄, AsX₅, ZnX₂, and HgX₂, where X is a halogen. Preferably, the Lewis acid catalyst is ZnCl₂ or HgBr₂.

In another aspect of the invention, a precursor of pentostatin or other heterocyclic compounds is provided that has the formula 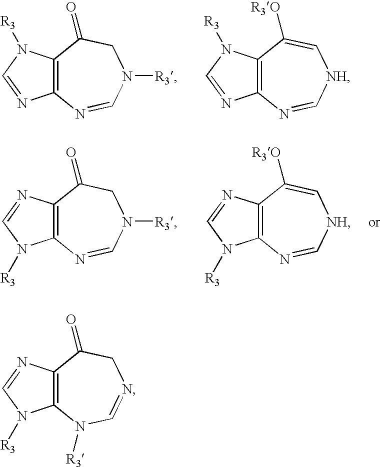
wherein R₃ and R₃′ are each independently H or a protective group, and at least one of R₃ and R₃′ is a protective group.

In yet another aspect of the invention, a method for manufacturing a precursor of pentostatin, diazepinone precursor 8a, is provided. In one embodiment, the method comprises: protecting hypoxanthine at one or more locations using a protecting group; reacting the protected hypoxanthine under a suitable condition in a appropriate solvent to yield a protected diazepinone precursor; precipitating the protected diazepinone precursor, and deprotecting the protected diazepinone precursor to yield the diazepinone precursor 8a.

In yet another aspect of the invention, a method for manufacturing pentostatin is provided. In one embodiment, the method comprises: protecting hypoxanthine at one or more locations using a protecting group; reacting the protected hypoxanthine under a suitable condition in an appropriate solvent to yield a protected diazepinone precursor; deprotecting the protected diazepinone precursor to yield the diazepinone precursor 8a; condensing the N-2 of the diazepinone precursor 8a with the C-1 position of 2-deoxy-D-ribose and derivatives to yield an intermediate 9a, and deprotecting and reducing the 8-keto functional group of the intermediate 9a to pentostatin.

In another embodiment, the method for manufacturing pentostatin comprises: protecting 2′-deoxyinosine at one or more locations using a protecting group; expanding the 6-member ring of the protected 2′-deoxyinosine to yield an intermediate 9a; and deprotecting and reducing the 8-keto functional group of the intermediate 9a to yield pentostatin, wherein R₁ and R₁′ are each independently a protective group.

In aspect of the invention, a method for manufacturing coformycin is provided. The method comprises:
protecting inosine at one or more locations using a protecting group;
expanding the O—C—N functionality of the 6-member ring of a protected inosine to yield an intermediate having the formula 22 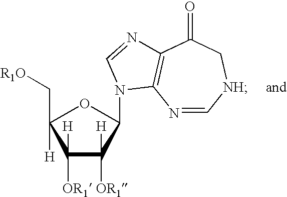
deprotecting and reducing the 8-keto functional group of the intermediate 22 to yield coformycin, wherein R₁, R₁′ and R₁″ are each independently H or a protective group.

The methods and compositions described above can also be used to synthesize and manufacture heterocyclic compounds other than pentostatin, especially pharmaceutically important heterocyclic compounds such as coformycin. Pentostatin, its precursors and derivatives may be used as therapeutic or diagnostics in the treatment of various diseases or conditions, such as hematological disorders, cancer, autoimmune diseases, and graft-versus-host disease.

### BRIEF DESCRIPTION OF THE DRAWINGS

FIG. 1A is a scheme for synthesis of a diazepinone precursor by Showalter and Baker (Chan, E.; Putt, S. R.; Showalter, H. D. H.; Baker, D. C. J. Org. Chem. 1982, 47, 3457-3464).

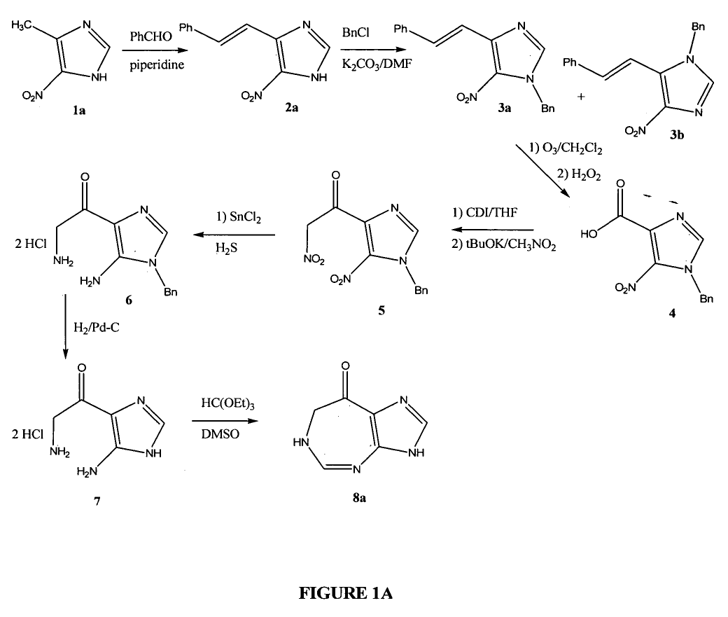

FIG. 1B is a scheme for glycosylation with peracylglycosyl chloride (Chan et al., supra)

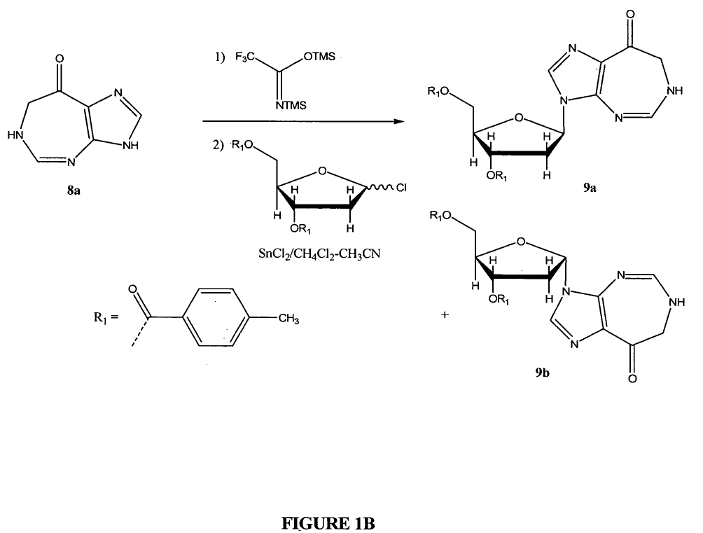

FIG. 1C is a scheme for non-stereocontrolled reduction (Chan et al., supra).

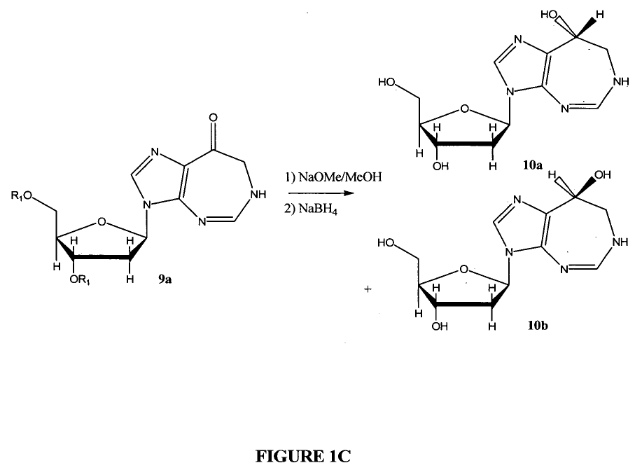

FIG. 2 is a scheme for synthesis of pentostatin improved by Chen (Chen, B.-C.; Chao, S. T.; Sundeen, J. E.; Tellew, J.; Ahmad, S. Tetrahedron Lett. 2002, 43, 1595-1596.).

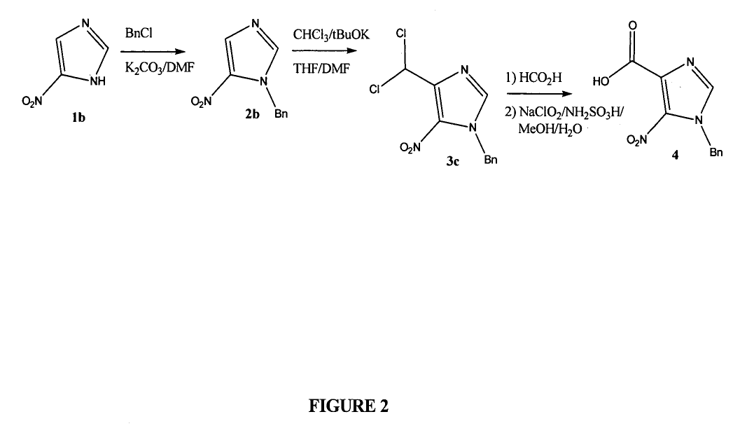

FIG. 3 is a scheme for stereocontrolled synthesis of the Cyclopentyl analogue of pentostatin (Ho, J. Z.; Mohareb, R. M.; Ahn, J. H.; Sim, T. B.; Rapoport, H. J. Org. Chem. 2003, 68,109-114).

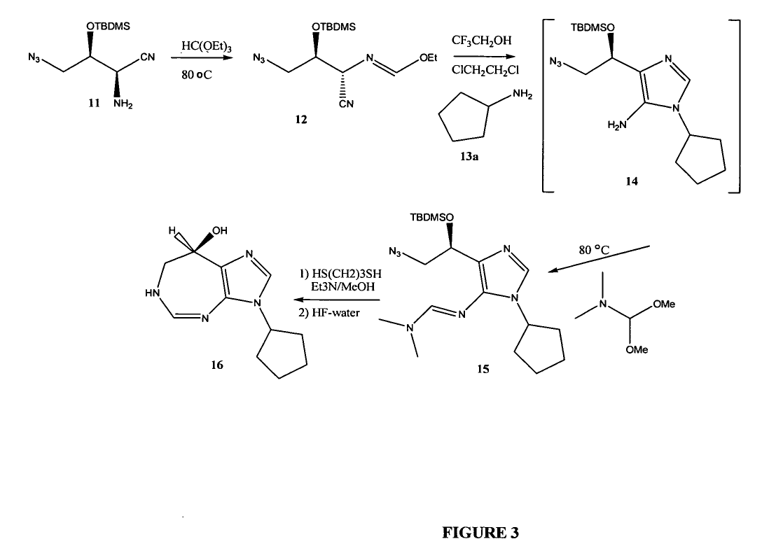

FIG. 4 illustrates the mechanism and intermediates for the biosynthesis of pentostatin by S. antibioticus, shown without phosphorylation.

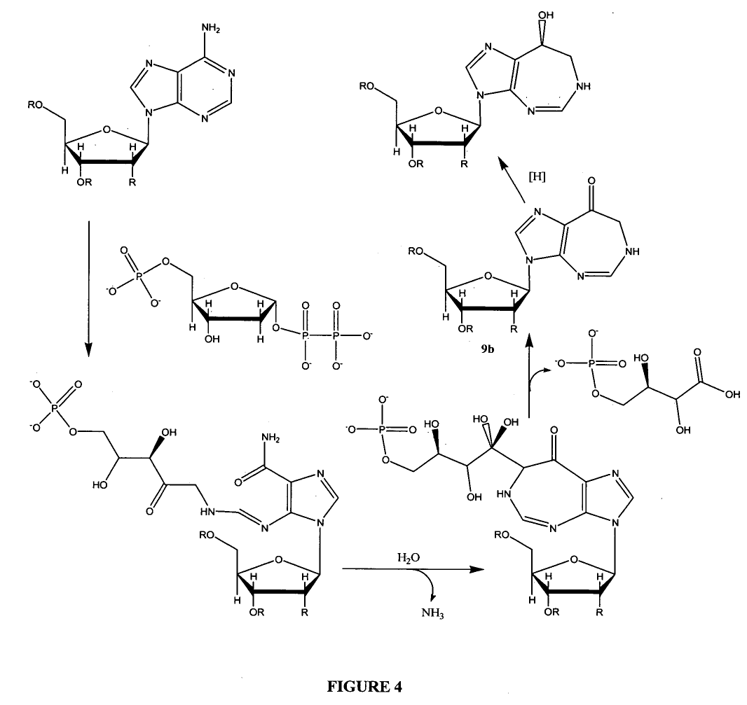

FIG. 5 is a scheme for introduction of R₂ protective group onto hypoxanthine, where R₂ can be any protective group.

FIG. 6A is a scheme for ring-expansion of protected hypoxanthines to diazepinone derivatives, where R₃ can be any protective group.

FIG. 6B shows a mass spectrum of isomeric mixture dibenzyl 6,7-dihyroimidazo[4,5-d][1,3]diazepin-8-(3H)-one with m/z=331 [M+H]⁺ and a doubly-repeated ring expanded impurity at with m/z=345 [M+H]⁺.

FIG. 6C shows a ¹H NMR of isomeric mixture of dibenzyl 6,7-dihyroimidazo[4,5-d][1,3]diazepin-8-(3H)-one, with DMSO at 2.50 ppm.

FIG. 6D shows the presence of the methylene (—CH2—) functional group of all three isomers of dibenzyl 6,7-dihyroimidazo[4,5-d][1,3]diazepin-8-(3H)-one at 4.12 to 3.92 ppm.

FIG. 6E shows a 400 MHz ¹H NMR spectrum of an isomeric mixture of dibenzyl hypoxanthines in d₆-DMSO.

FIG. 6F is an expanded view of a 400 MHz ¹H NMR spectrum of three isomeric dibenzyl hypoxanthines in d₆-DMSO between 6.0 to 2.0 ppm.

FIG. 6G shows the dependence of the magnitude of the geminal coupling constant on the HCH angle.

FIG. 6H lists samples of or ²J coupling constants.

FIG. 6I shows a 400 MHz ACD/HNMR spectrum of dibenzyl 6,7-dihyroimidazo[4,5-d][1,3]diazepin-8-(3H)-one in non-polar and non-aromatic solvent.

FIG. 6J is an expanded view of a 400 MHz ACD/HNMR spectrum of dibenzyl 6,7-dihyroimidazo[4,5-d][1,3]diazepin-8-(3H)-one between 4.30 to 3.90 ppm, where the cyclic—CH₂— group appears, in non-polar and non-aromatic solvent.

FIG. 7 shows a scheme for deprotection of protected diazepinones to diazepinone 8a.

FIG. 8 shows a scheme for modifications of the condensation condition to improve handling and scalability.

FIG. 9 shows a synthetic scheme for improving efficiency and yield of diazepinone condensation with 2-deoxy-D-ribose.

FIG. 10 is a scheme for improved deprotection and other asymmetric reductions of the 8-keto functional group to pentostatin.

FIG. 11 is an example of a variation on the synthesis of pentostatin by ring expansion.

FIG. 12 is another example of a variation on the synthesis of pentostatin by ring expansion.

FIG. 13 is another example of a variation on the synthesis of pentostatin by ring expansion.

FIG. 14 is an example of synthesis of a variation on the synthesis of coformycin by ring expansion.

### DETAILED DESCRIPITION OF THE PRESENT INVENTION

The present invention provides novel compositions and methods for efficiently preparing and manufacturing pentostatin. Also provided are novel precursors of pentostatin, pentostatin analogs and derivatives.

In one aspect of the invention, a method is provided for a total chemical synthesis of pentostatin via a route of heterocyclic ring expansion. Specifically, a heterocyclic pharmaceutical intermediates for drugs such as pentostatin, e.g., the diazepinone precursor 8a and intermediate 9a, can be obtained efficiently through a ring expansion of an O—C—N functionality in a hypoxanthine derivative or a protected 2′-deoxyinosine. The inventors believe that the ring expansion is a very efficient route for chemical transformation from a readily available, economic starting material to a complex, active pharmaceutical molecule. By using this ring expansion method, the diazepinone precursors 8a and 9a, which are important precursor and intermediate of pentostatin, can be obtained by no more than three synthetic steps, which tremendously reduces the total number of steps necessary to synthesize pentostatin. The diazepinone precursor 8a can be condensed with 2-deoxy-D-ribose (or other derivatives) in various ways and intermediate 9a reduced to yield pentostatin (or other derivatives or analogs of pentostatin).

It should be noted that the methodology and precursors provided herein can also be applied to the synthesis of compounds containing other heterocycles that require expansion of the heterocyclic ring at an O—C—N functionality, e.g., synthesis of heterocycles starting from inosine and 2′-deoxyinosine.

In some embodiments of the present invention, synthesis of pentostatin includes three parts: Module A—preparation of a diazepinone base; Module B—condensation of heterocyclic base with 2-deoxy-D-ribose; and Module C—formation of 8-(R)-hydroxyl group. According to these embodiments, there are three modular variations in the process of synthesizing pentostatin: ABC; ACB; and BAC. It should be noted that any variations based on these modules are within the scope of the present invention. The foundation of each variation is ring expansion of the O—C—N functionality of a hypoxanthine, 2′-deoxyinosine, or inosine into a cyclic α-aminoketone diazepinone. Each variation offers distinct advantages but may vary in the degrees of overall robustness, efficiency, yield and economy.

### 1. Module A—Synthesis of diazepinone precursor 8a (6,7-Dihyroimidazo[4,5-d][1,3]diazepin-8-(3H)-one)

The present invention provides an effective chemical method for synthesizing the key intermediate of pentostatin, diazepinone precursor 8a. As outlined below, the diazepinone precursor 8a can be obtained via a route of ring expansion of hypoxanthine.

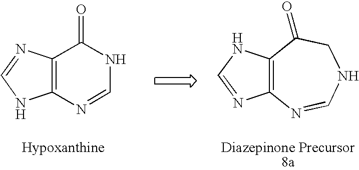

Hypoxanthine Diazepinone Precursor 8a In general, the ring expansion of hypoxanthine may be achieved by direct insertion of a methylene function with a reagent such as diazomethane, which can be photocatalyzed (Doering, W. von E.; Knox, L. H. J. Am. Chem. Soc. 1951, 75, 297-303) or Lewis acid-catalyzed (Wittig, von G.; Schwarzenbach, K. Liebigs Ann. Chem. 1961, 650, 1-21). Preferably, trimethylsilyl diazomethane is used since it is a more stable reagent and may offer a safer alternative to volatile diazomethane (Seyferth, D.; Menzel, H.; Dow, A. W.; Flood, T. C. J. Organometallic Chem. 1972, 44, 279-290).

According to the present invention, diazomethane and trimethylsilyl diazomethane are utilized to expand a heterocyclic ring containing an O—C—N functionality. The ring expansion chemistry of the present invention is applicable to all suitably protected O—C—N and cyclic O—C—N. In addition, the ring expansion can be controlled specifically and kinetically to yield only the required number of ring expansion, which is accomplished by incorporating appropriate protection groups and under a suitable solvent condition.

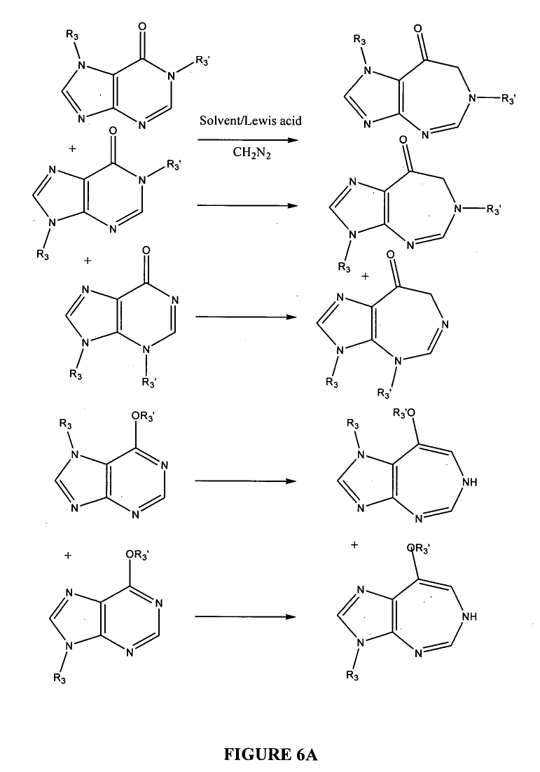

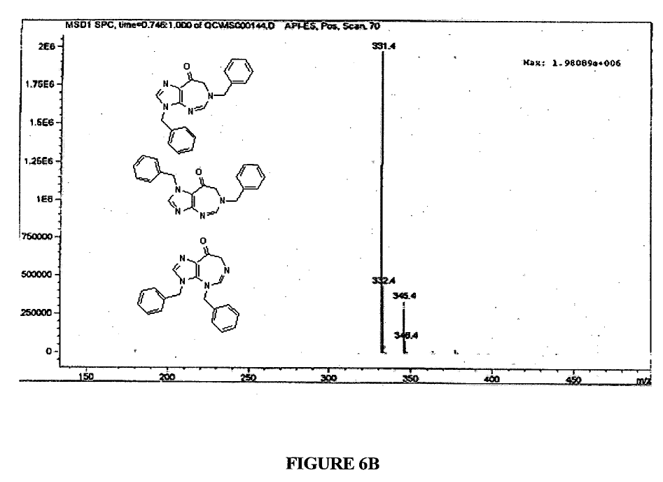

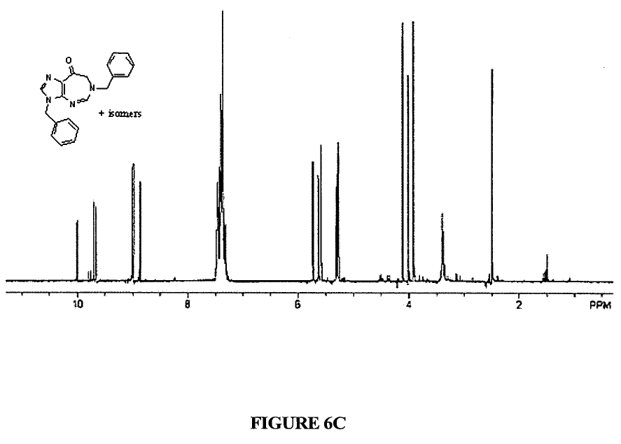

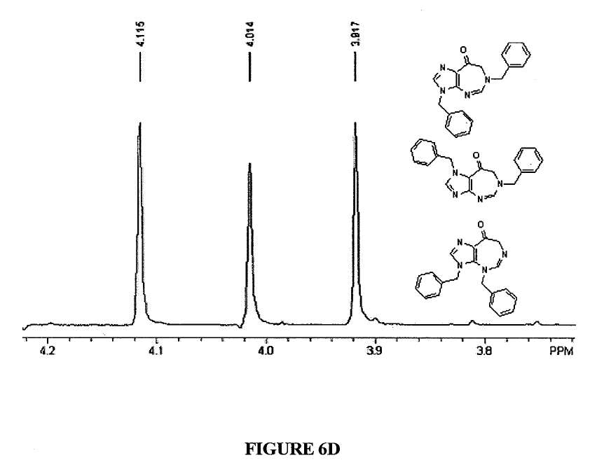

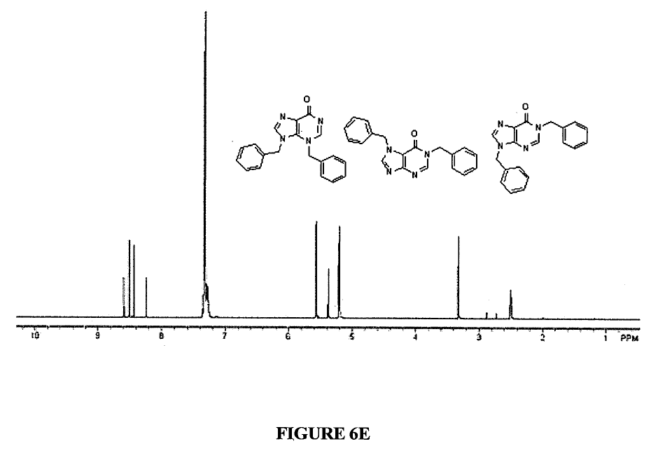

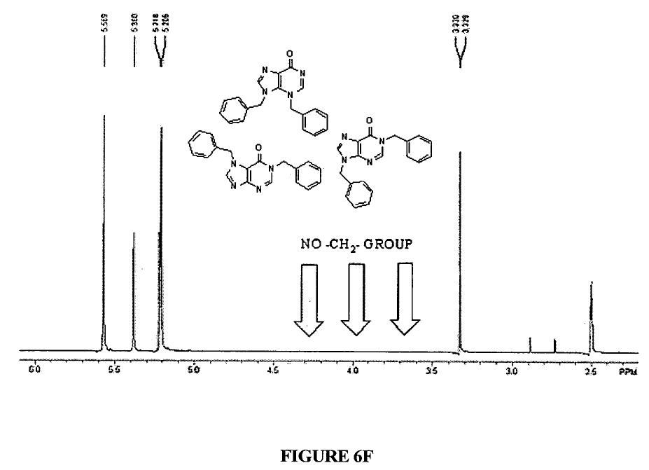

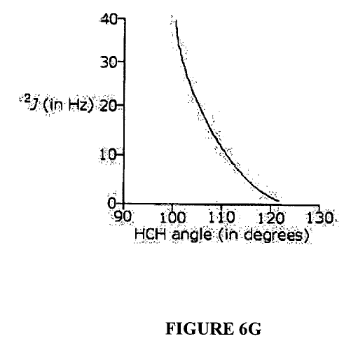

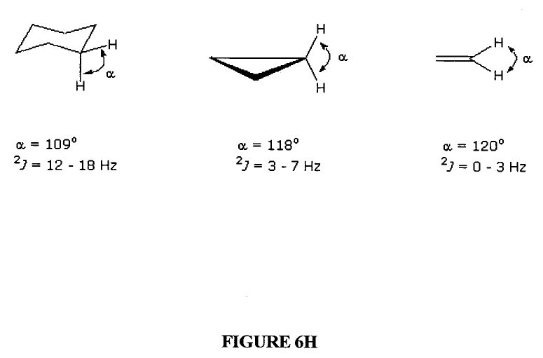

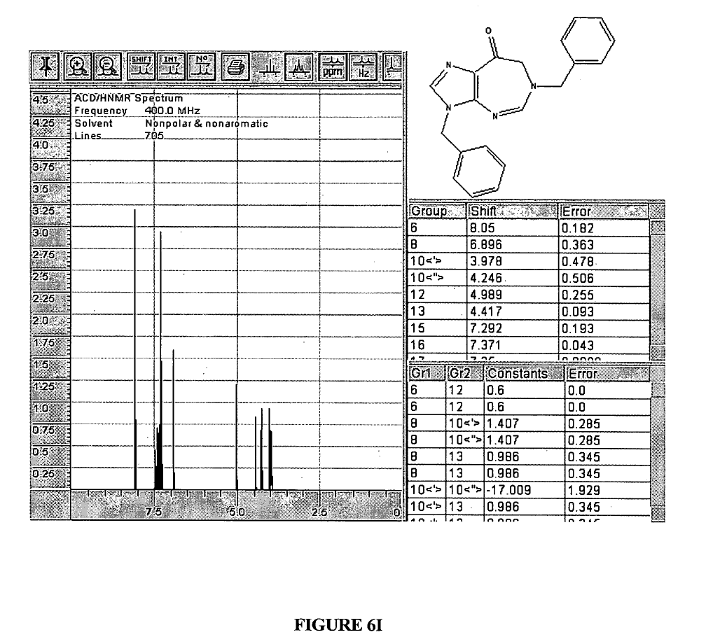

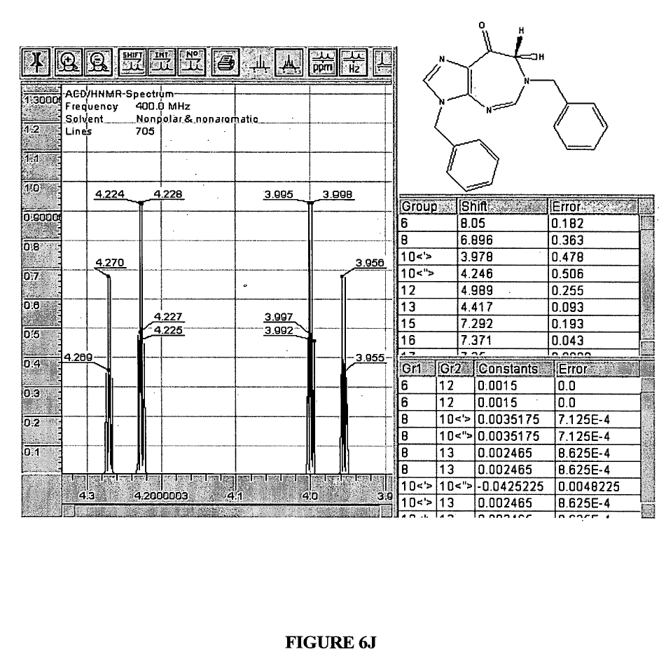

In one embodiment, the method of synthesizing diazepinone precursor 8a involves treating a suitably protected hypoxanthine derivative, in a solution of an organic solvent with a Lewis acid catalyst and under anhydrous atmosphere, with a freshly prepared anhydrous solution of diazomethane in ether. FIG. 6A illustrates examples of such a synthesis scheme. The reaction occurs at −78° C. to 25° C. for a period of a few minutes to a few hours.

With regard to the starting material, hypoxanthine, 2′-deoxyinosine or inosine, the heterocyclic hypoxanthine ring does not contain any stereocenter. It is commercially available from such sources as Alfa Aesar, a Johnson Matthey Co., Ward Hill, Mass. and Aldrich Chemical Co., Milwaukee, Wis. While any purity of hypoxanthine, 2′-deoxyinosine, and inosine can be used, at least about 92% purity is preferable. The amount of hypoxanthine, 2′-deoxyinosine, or inosine used can be any amount, as long as there are sufficient amounts of the protective groups and a base effective in assisting the formation of a nucleophilic hypoxanthine ion ring to make protected hypoxanthine derivatives.

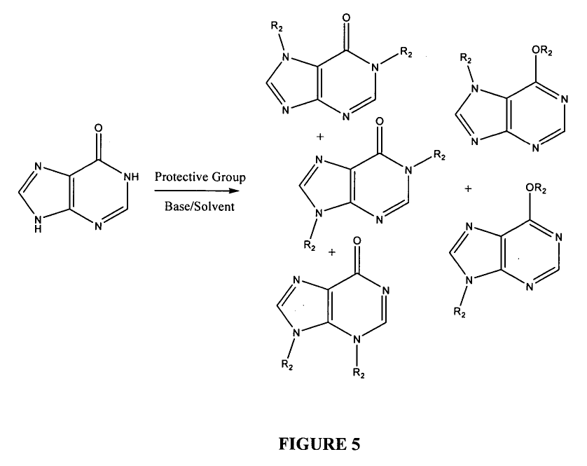

The base can be any base that is capable of forming a nucleophilic hypoxanthine ion or inducing the protective reagent in a matter that results in the protection of the imidazole secondary amine and the O—C—N functionality. The protection occurs at least on the imidazole secondary amine. Examples of bases include, but are not limited to: pyridine; aqueous NaOH; NEt₃; DMAP; K₂CO₃; Na₂CO₃; NaH; Na/NH₃; MeLi; and t-BuOK. FIG. 5 shows a scheme for introduction of R₂ and R₂′ protective group onto hypoxanthine, where R₂ and R₂′ can be any protective groups and can be the same or different from each other.

The protective groups are all groups that are capable of forming a covalent bond with alt imidazole secondary amines, cyclic amine and the O—C—N(O═C—NH⇄HO—C═N) functionality in the hypoxanthine ring, thereby protecting hypoxanthine ring of hypoxanthine, 2′-deoxyinosine, and inosine by any combination and variation thereof. Added features of all applicable protective groups are that they 1) help to make the hypoxanthine, 2′-deoxyinosine, and inosine derivative to be more soluble in an organic solvent, 2) allow the ring-expansion to proceed without side-reaction and decomposition (i.e. they are stable under the condition of diazomethane and a Lewis acid), and 3) assist isolation and purification of the diazepinone without interfering with the ring-expansion. Examples of protective groups for the imidazole secondary amine and the O—C—N functionality include, but are not limited to: carbamates (i.e. methyl, ethyl, t-butyl, benzyl, 9-fluorenylmethyl, 2,2,2-trichloroethyl, 1-methyl-1-(4-biphenyl)ethyl, and 1-(3,5-di-t-butyl)-1-methylethyl); amides (i.e. acetamide, trifluoroacetamide, and benzamide); aryl amines (i.e. benzylamine, 4-methoxybenzylamine, and 2-hydroxybenzylamine); and silyl amines.

With respect to the Lewis acid used for ring-expansion, the acid is effective in catalyzing the reaction specifically at the C—N bond of the O—C—N functionality, which inserts a methylene group between the O—C—N bond to form a separate ketone functionality and an amine functionality, forming the so called α-aminoketone. Examples of acids include, but are not limited to: trimethylsilyl triflate (TMSOTf); BX₃; AlX₃; FeX₃; GaX₃; SbX₅; SnX₄; AsX₅; ZnX₂; and HgX₂, where X is a halogen. The amount of Lewis acid used in the reaction should be in the range of 1% to 200% stoichiometric equivalents.

With respect to the organic solvent, the solvent is effective in solubilizing the starting material for the reaction to progress without hindrance and in a timely matter, and it should not hinder isolation and purification. Preferably, these organic solvents should be restricted to solvents acceptable for pharmaceutical processing, which include, but are not limited to: acetonitrile; chlorobenzene; dichloromethane; methylcyclohexane; N-methylpyrrolidone; nitromethane; acetone; DMSO; ethyl acetate; ethyl ether; and ethyl formate.

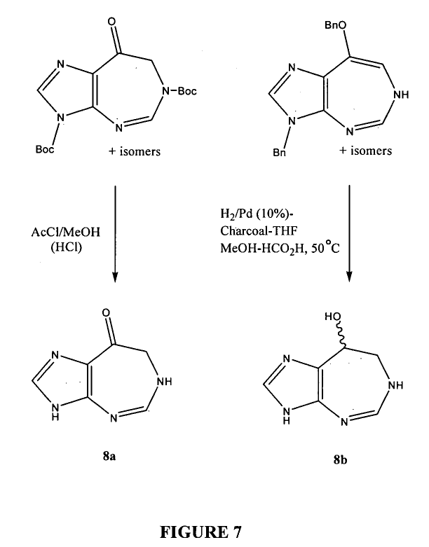

In one embodiment, the protected hypoxanthine, 2′-deoxyinosine, or inosine is added to a reaction vessel with a stirring bar for mixing before adding the selected organic solvent and Lewis acid, while maintaining anhydrous atmosphere. The stirred mixture is clear and homogeneous at −78° C. to 25° C. While maintaining anhydrous atmosphere, enough freshly prepared diazomethane in ether is slowly added to prevent over-bubbling and addition of too much diazomethane; the mixture is continual stirred for a period of a few minutes to a few hours until the protected hypoxanthine, 2′-deoxyinosine, or inosine is completely consumed, as determined by TLC or HPLC. Once the protected hypoxanthine, 2′-deoxyinosine, or inosine is completely consumed, the protected diazepinone has already started precipitating out of the reaction mixture; an anti-solvent, preferably a solvent acceptable for pharmaceutical processing similar to those described above, is added to further precipitate the ring-expanded product, the desired diazepinone derivative. The diazepinone derivative may be purified by a suitable recrystallization solvent or SiO₂ flash column chromatography. Each diazepinone derivative is then subjected to a deprotection procedure specific to its chemistry to give diazepinone precursor 8a and intermediate 9a (e.g., FIG. 7).

Two factors may be considered determining which deprotection procedure is used: yield of diazepinone and scalability. A harsh deprotection procedure would be accompanied by significant decomposition of the diazepinone, especially if it required a prolonged period of time. Under certain conditions, the yield may be excellent at microscale but poor at grams and kilograms scales.

### 2. Module B—Synthesis of Compound 9a (3-[2-Deoxy-β-D-erythro-pentofuranosyl]-6,7-dihydroimidazo[4,5-d][1,3]diazepin-8 (3H)-one)

Once the diazepinone precursor 8a has been synthesized, a synthetic scheme is provided that improves the procedure of Showalter and Baker described above in the section of “Description of Related Art”, especially with improved handling, efficiency, scalability, and yield.

In the Showalter and Baker procedure, a pertrimethylsilylated diazepinone derivative was condensed to the 2-deoxy sugar via a peracylglycosyl chloride adapted from the stannic chloride catalyzed process of Vorbrüggen at the low temperature of −35° C. in the toxic solvent 1,2-dichloroethane, which is not a pharmaceutically acceptable solvent based on its high toxicity and should be avoided.

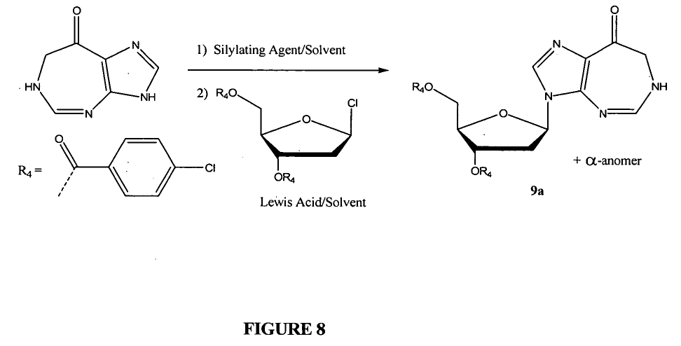

To improve this condensation, according to the present invention, weaker Lewis acids (which include, but are not limited to: ZnCl₂ and HgBr₂) may be employed. Pharmaceutically acceptable solvents (which include, but not limited to: toluene and tetrahydrofuran) that allow the condensation to occur only slowly at low temperatures should work well at elevated temperatures (0° C. to 50° C.), which should improve scalability and handling. FIG. 8 shows an embodiment of the improved condensation procedure. The α/β mixture can be separated by fractional crystallization.

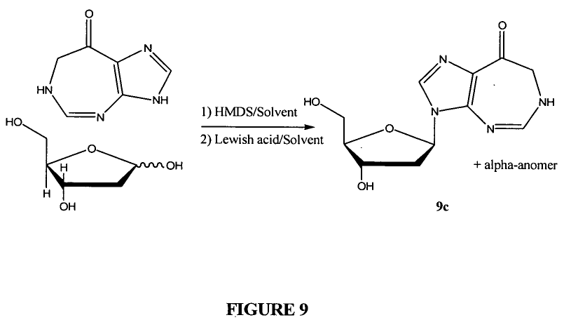

To further improve the diazepinone condensation with 2-deoxy-D-ribose, a one-pot synthesis of free nucleoside by Vorbrüggen (Bennua-Skalmowski, B.; Krolikiewicz, K.; Vorbrüggen, H. Tetrahedron Lett. 1995, 36, 7845-7848) is adapted to directly make free 6,7-dihyroimidazo[4,5-d][1,3]diazepin-8-(3H)-one (FIG. 9, compound 9c).

Persilylation of excess 2-deoxy-D-ribose and diazepinone 8a, 2′-deoxyinosine, and inosine could be accomplished with a variety of silylating agents (which include but are not limited to: hexamethyldisilazane (HMDS); trimethylchlorosilane; bromotrimethylsilane; N-(trimethylsilyl)acetamide; bis-(trimethylsilyl)trifluoroacetamide; trimethylsilyl trifluoroacetate; trimethylsilyl triflate; and any combination thereof) in pharmaceutically acceptable solvents (which include, but not limited to: acetonitrile and tetrahydrofuran, respectively) for 3 hours at reflux.

Persilylation of excess 2-deoxy-D-ribose and diazepinone 8a, and evaporation followed by condensation in presence of Lewis acid in pharmaceutically acceptable solvents (which include, but are not limited to: acetonitrile and tetrahydrofuran, respectively) with 1.1 equivalents of a Lewis acid (which include, but are not limited to: TMSOTf, BX₃, AlX₃, FeX₃, GaX₃, SbX₅, SnX₄, ASX₅, ZnX₂, and HgX₂, where X is a halogen), and transsilylation with methanolic base (which include, but not limited to: NaHCO₃ or NH₃ in methanol) should furnish free 9c, which should be separable from the α-anomer with fractional crystallization.

In FIG. 8, the protection shown on 2-deoxy-D-ribose is p-toluoyl, which is just one example. Protection group R₄ includes, but are not limited to: ethers (such as methoxymethyl, benzyloxymethyl, allyl, propargyl, p-chlorophenyl, p-methoxypehenyl, p-nitrophenyl, benzyl, p-methoxybenzyl, dimethoxybenzyls, nitrobenzyl, halogenated benzyls, cyanobenzyls, trimethylsilyl, trimethylsilyl, triisopropylsilyl, tribenzylsilyl, and alkoxysilyls); esters (such as the variety of acetates and benzoates); carbonates (such as methoxymethyl, 9-fluorenylmethyl, 2,2,2-trichloroethyls, vinyl, allyl, nitrophenyls, and benzyls); sulfonates (such as allylsulfonate, mesylate, benzylsulfonate, and tosylate); cyclic acetals and ketals (such as methylene, ethylidene, acrolein, isopropylidene, cyclopentylidene, cyclohexylidene, cycloheptylidene, the variety of benzylidenes, mesitylene, 1-naphthaldehyde acetal, benzophenone ketal, o-xylyl ether); chiral ketones (such as camphor and menthone); cyclic ortho esters (such as methoxymethylene, ethoxymethylene, 1-methoxyethylidene, methylidene, phthalide, ethylidene and benzylidene derivatives, butane-2,3-bisacetal, cyclohexane-1,2-diacetal, and dispiroketals); silyl derivatives (such as di-t-butylsilylene and dialkylsilylene groups); cyclic carbonates; cyclic borates; and combinations and variations thereof.

### 3. Module C—Synthesis of Pentostatin ((8R)-3-(2-deoxy-β-d-erythro-pentofuranosyl)-3,6,7,8-tetrahydroimidazo[4,5-d][1,3]diazepin-8-ol)

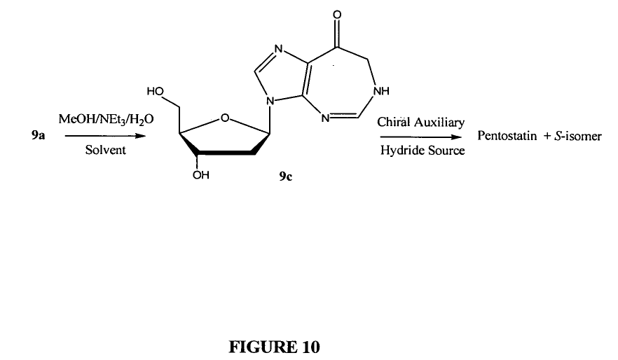

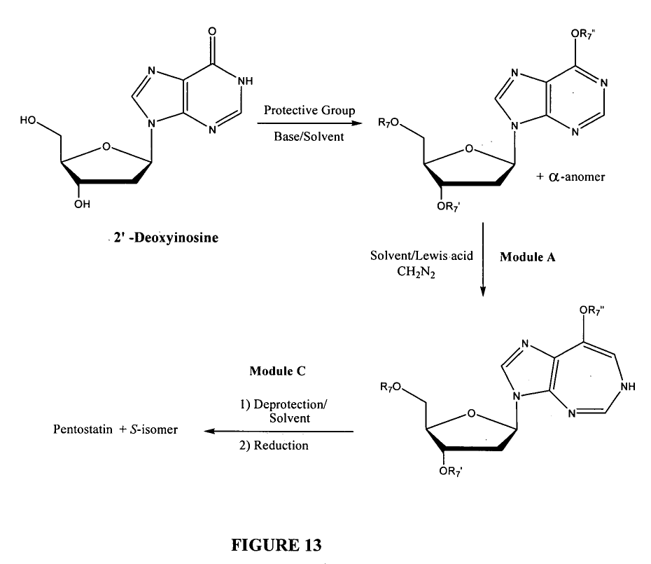

Before proceeding to reduction of the 8-keto group, if there are protective groups, they could be removed. In the Showalter and Baker procedure, methanolic sodium methoxide was used, which is a harsh environment and may lead to decomposition of the diazepinone moiety. A milder deprotection procedure (Phiasivongsa, P.; Gallagher, J.; C. Chen; P. R. Jones; Samoshin, V. V., Gross, P. H. Org. Lett. 2002, 4, 4587-4590) is more preferred in order to minimize decomposition and increase the yield of free 6,7-dihyroimidazo[4,5-d][1,3]diazepin-8-(3H)-one (compound 9c) before reduction (FIG. 10). To further improve the preparation of intermediate like intermediate 9c, the synthesis can begin with commercially available 2′-deoxyinosine as shown in FIG. 13.

Showalter and Baker tried a variety of sterically hindered borohydrides, but the best yielding process was determined to be the non-selective reductions with sodium borohydride and nickel catalyzed hydrogenation. Chan, E.; Putt, S. R.; Showalter, H. D. H.; Baker, D. C. J. Org. Chem. 1982, 47, 3457-3464. FIG. 10 shows a scheme for an improved method of deprotection and other asymmetric reductions of the 8-keto functional group to pentostatin.

In the present invention, hydrides doped with chiral auxiliaries are preferably implemented. Examples of hydrides include, but are not limited to, NaBH₄; LiAlH₄; BF₃-THF; and LiBH₄. Examples of chiral auxiliaries include, but are not limited to, N,N′-dibenzoylcystine; K glucoride; B-chlorodiisopinocampheyborane; [(1S)-endo]-(−)-bomeol; and (S)-(+)- and (R.)-(−)-2-aminobutan-1-ol).

The presence of the diazepinone moiety in free 6,7-dihyroimidazo[4,5-d][1,3]diazepin-8-(3H)-one (9c) and protected derivatives like 9a makes them good α-aminoketone candidates. This approach has been shown to work well with α-aminoketones. Asymmetric reductions of α-aminoketones with LiAH₄ treated with (S)-(+) or (R.)-(−)-2-(2-isoindolinyl)butan-1-ol (which were easily prepared in one step from commercially available (S)-(+)- and (R.)-(−)-2-aminobutan-1-ol, respectively, in high yields) to aminoalcohols with enantiomeric excess in the range of 40-97% have been achieved. Brown, E.; Leze, A; Touet, J. Tetrahedron: Asymmetry 1996, 7, 2029-2040. The inventors believe that asymmetric reductions of this type, those that have been shown to be successful for α-aminoketones, should improve the yield of pentostatin.

In addition, reduction of the 8-keto functional group may also achieved with economical hydrides (which include but are not limited to: KBH₄; NaBH₃CN; MgH₂; borohydride on Montmorillonite-KSF support; and borohydride on Amberlite® support), metals (which include but are not limited to: Li, Na or K/NH₃; Li, Na or K/alcohol; H₂ and nickel catalysts such as nickel boride and Raney nickel; H₂ and platinum catalysts; H₂ and iron catalysts such as FeCl₂; and Fe/acetic acid), and titanocene-catalyzed reduction with water and metal dust (which includes but is not limited to: zinc and manganese).

Based on the above description, a total synthesis of pentostatin may be achieved through combination of the modules A, B and C in any order. A particular synthetic pathway is modular ABC. Two other variations that are also preferred include modular ACB and BAC, which are described as follows.

4. Modular ACB—Total Synthesis of Pentostatin

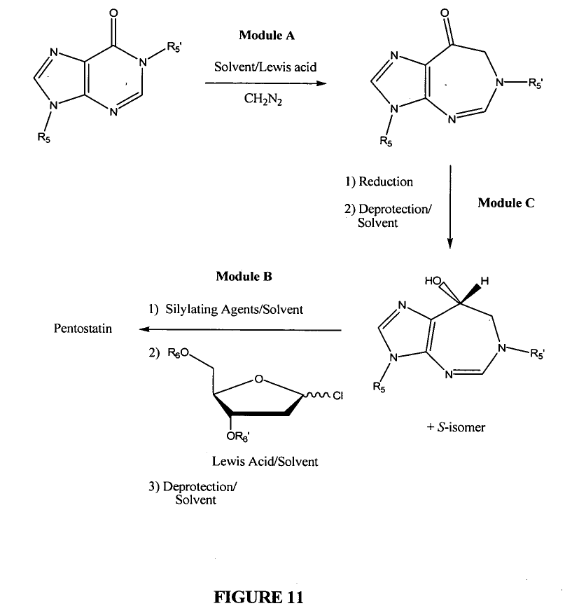

FIG. 11 shows an example of a variation on the total synthesis of pentostatin by ring expansion, where R₅ and R₅′ may be any protective group and may be the same or different from each other; and R₆ and R₆′ may be any protective group and may be the same or different from each other.

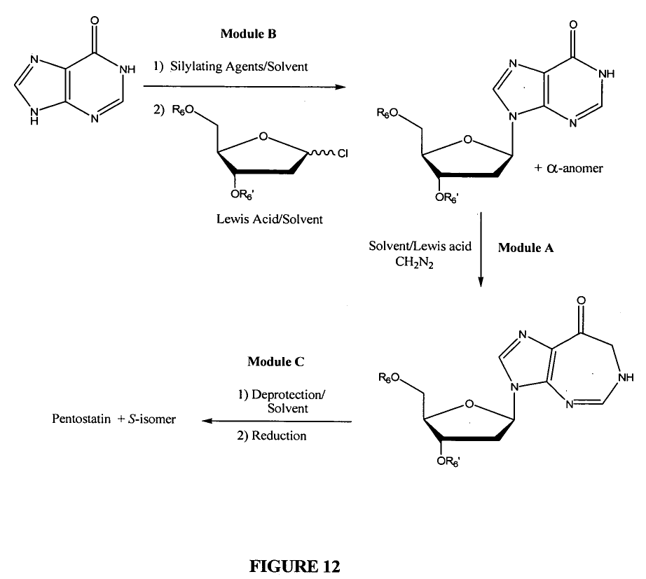

In FIGS. 10 and 12, although deprotection is performed before reduction, the reverse sequence, where the protection is left in place during reduction and then removed, could be more desirable. The different protections on the sugar (see discussion of FIG. 8) allow the deprotection and reduction sequence to be performed in either way. In FIG. 11, it was sometimes desirable to perform reduction before removal of the protective groups, for reasons relating to handling, yield and purification. Then, it was necessary to remove the protective groups before condensation. After condensation, the sugar protective groups had to be removed to give the final product, pentostatin.

5. Modular BAC—Total Synthesis of Pentostatin

FIG. 12 shows another example of a variation on the synthesis of pentostatin by ring expansion, where R₆ and R₆′ may be any protective group and may be the same or different from each other.

6. Modular AC—Total Synthesis of Pentostatin

FIG. 13 shows another example of a variation on the synthesis of pentostatin by ring expansion, where R₇, R₇′ and R₇″ may be any protective group and may be the same or different from each other. Because the synthesis begins with commercially available 2′-deoxyinosine, the total synthesis of pentostatin is further shortened by bypassing at a couple of synthetic steps.

Examples of protective groups R₇ and R₇′ for the hydroxyl groups in the 2-deoxyribose ring include, but are not limited to: benzyl ethers (e.g., p-methoxybenzyl, 3,4-dimethoxybenzyl, nitrobenzyl, and p-cyanobenzyl); silyl ethers (e.g., triakylsilyl and alkoxydialkylsilyl); and esters (e.g. acetate, halogenatedacetate, alkoxyacetate, and benzoate). Examples of protective group R₇″ for the oxygen on the heterocyclic hypoxanthine ring include, but are not limited to: benzyl ethers (e.g., p-methoxybenzyl, 3,4-dimethoxybenzyl, nitrobenzyl, and p-cyanobenzyl) and silyl ethers (e.g., triakylsilyl and alkoxydialkylsilyl). Alternatively, the NH amide of the heterocyclic ring could be protected via transformation (NH→N—R₇′″ as shown below) into carbamates (i.e. methyl, ethyl, t-butyl, benzyl, 9-fluorenylmethyl, 2,2,2-trichloroethyl, 1-methyl-1-(4-biphenyl)ethyl, or 1-(3,5-di-t-butyl)-1-methylethyl carbamate) as follows:

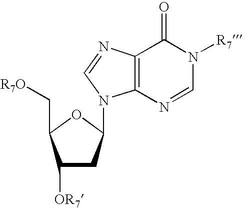

Deprotection of the benzyl ethers could be achieved with mild reagents such as the following: PhSTMS, ZnI₂, tetrabutylammonium iodide, 1,2-dichloroethane at 60° C. for 2 hours; rhodium/Al₂O₃/H₂; and Ph₃C⁺BF₄⁻ in dichloromethane. The ester protective groups could be cleaved with basic methanol and alcohols (e.g. ammonia/methanol and sodium methoxide). The silyl ethers could be easily removed with the following reagents: tetrabutylammonium fluoride in tetrahydrofuran; citric acid in methanol at 20° C.; FeCl₃ in acetonitrile at ambient temperature; and BF₃-etherate. Carbamates such as the Boc could be easily removed as described in Example 2.

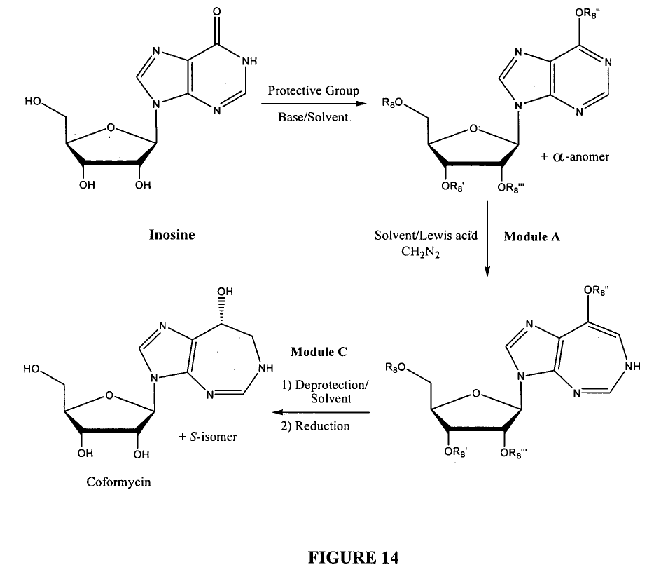

FIG. 14 shows an example of how other pentostatin derivatives, such as the synthesis of coformycin, could be achieved by ring expansion, where R₈, R₈′, R₈″ and R₈′″ may be any protective group and may be the same or different from each other. Because the synthesis begins with commercially available inosine, the total synthesis of coformycin is further shortened by bypassing a couple of synthetic steps.

These shortened synthesis processes of pentostatin, its analogs and derivatives are highly desirable, considering the overall robustness, efficiency, yield and economy.

The following example serves to more fully describe the manner of using the above-described invention. It is understood that the example in no way serves to limit the scope of this invention, but rather is presented for illustrative purpose. All references cited herein are incorporated by reference in their entirety.

### EXAMPLES

1. Syntheis of Pentostatin from Dibenzyl Hypoxanthine

According to the present invention, pentostatin can be synthesized through the route of ring-expansion of protected hypoxanthines to generate diazepinone derivatives as outlined in FIG. 6A where R₃ and R₃′ can be any protective group and can be the same or different from each other. In this example, pentostatin is synthesized via ring-expansion of dibenzyl-protected hypoxanthine.

To a solution containing 16 mL of water and 10 g KOH in a three-neck 500 mL round bottom flask was added diethylene glycol monomethyl ether (28 mL). The flask was fitted with a simple distillation unit with a water condenser and a 250 mL receiving round bottom flask immersed in an ice-bath. A 100 mL dropping funnel-containing 10 g of Diazald dissolved in ether (90 mL) was also fitted. The one unused neck of the receiver was closed with a rubber septum and balloon filled with nitrogen. The water was turned on and the distilling flask was slowly heated in an oil bath (75-80° C.) while slowly adding the Diazald solution. The rate of addition should be equal to rate of distillation, which should take about 20 min. When all the Diazald was used up, an additional 10 mL of ether was added and continue until distillate was clear. The ether should contain about 30 mmol of diazomethane.

An isomeric mixture of dibenzyl hypoxanthine (0.5 g, 1.58 mmol) was dissolved in anhydrous dichloromethane (50 mL) in a 250 mL round bottom flask before boron trifluoride diethyl etherate (0.4 mL, 2.6 mmol) was added. The mixture was closed with a rubber septum under N₂-atmosphere and cooled in an ice bath before the freshly prepared diazomethane etherate (50 mL) was slowly added via a syringe to prevent over bubbling and addition of too much diazomethane. The mixture was stirred in the ice bath for about 30 minutes under N₂-atmosphere for the reaction to be completed, as indicated by TLC (1:1:0.2 petroleum ether-ethyl acetate-methanol), at which point the product could be seen precipitating. Anhydrous diethyl ether (200 mL) was added to completely precipitate the product, and then the flask was flushed with N₂-gas, closed with a rubber septum and stored in the freezer (0° C.) overnight (12 hours). The white solid, a mixture of three isomers of dibenzyl 6,7-dihyroimidazo[4,5-d][1,3]diazepin-8-(3H)-one, was filtered, rinsed with diethyl ether (50 mL) and dried in vacuo for at least 6 hours.

The exact mass of the three isomers of dibenzyl 6,7-dihyroimidazo[4,5-d][1,3]diazepin-8-(3H)-one is 330.148 Daltons compared to 316.132 Daltons for their respective dibenzyl hypoxanthine, the difference of gaining a methylene (—CH₂—) functional group. FIGS. 6B and 6C show an API-ES mass spectrum and a 400 MHz ¹H NMR spectrum (d₆-DMSO) of the crude isomeric mixture of dibenzyl 6,7-dihyroimidazo[4,5-d][1,3]diazepin-8-(3H)-ones, contaminated with an 8-member ring β-aminoketone from doubly-repeated ring expansion, which gained two methylene (—CH₂—) functional groups. FIG. 6B shows a mass spectrum of isomeric mixture dibenzyl 6,7-dihyroimidazo[4,5-d][1,3]diazepin-8-(3H)-one with m/z=331 [M+H]⁺ and a doubly-repeated ring expanded impurity at with m/z=345 [M+H]⁺. FIG. 6C shows ¹H NMR of isomeric mixture of dibenzyl 6,7-dihyroimidazo[4,5-d][1,3]diazepin-8-(3H)-one, with DMSO at 2.50 ppm. However, the β-aminoketone impurity could be removed by a variety of purification procedures (which include, but are not limited to: fractional recrystallization; column chromatography; and preparative HPLC) or prevented from forming in the first place by controlling the reaction condition to precipitate only the desired diazepinone. FIG. 6D shows the presence of the methylene (—CH2—) functional group of all three isomers of dibenzyl 6,7-dihyroimidazo[4,5-d][1,3]diazepin-8-(3H)-one at 4.12 to 3.92 ppm. FIG. 6E shows a 400 MHz ¹H NMR spectrum of an isomeric mixture of dibenzyl hypoxanthines in d₆-DMSO. FIG. 6F shows an expanded view of a 400 MHz ¹H NMR spectrum of three isomeric dibenzyl hypoxanthines in d₆-DMSO between 6.0 to 2.0 ppm.

FIG. 6C and the selected region of 4.2 to 3.8 ppm in FIG. 6D clearly show the presence of the three distinct newly formed methylene (—CH₂—) singlets for the three isomers at 4.12, 4.01, and 3.92 ppm, which are obviously absent in the starting isomeric mixture of dibenzyl hypoxanthines as shown in FIG. 6E and the selected region of 4.5 to 3.5 ppm in FIG. 6F. The expanded view region between 6.5 and 2.0 ppm (FIG. 6F) shows only DMSO at 2.50 ppm, solvent impurities around 3.33 pm, and benzylic hydrogen nuclei (Ph-CH₂—) from 5.57 to 5.21 ppm.

In the literature (Chan, E.; Putt, S. R.; Showalter, H. D. H.; Baker, D. C. J. Org. Chem. 1982, 47, 3457-3464), a 200 MHz ¹H NMR spectrum of unprotected diazepinone 8a in d₆-DMSO had the methylene (—CH₂—) functional group appearing as a singlet at 4.37 ppm, which is close to those of dibenzyl 6,7-dihyroimidazo[4,5-d][1,3]diazepin-8-(3H)-ones. However, the splitting pattern and chemical shifts of the unprotected diazepinone 8a may not be fully representative of dibenzyl 6,7-dihyroimidazo[4,5-d][1,3]diazepin-8-(3H)-ones. The methylene (—CH₂—) hydrogen nuclei are expected to be diastereomeric and should exhibit a set of double doublet peaks due to geminal coupling (also called two-bond coupling or ²J) since they are part of a heterocyclic ring, and therefore very likely to experience different electronic environment. Geminal coupling constants can be large, which range from +42 to −20 Hz with typical values being around 10 to 20 Hz, and dependent upon the —CH₂— bond angle (FIGS. 6G and 6H), influence of neighboring π bonds, ring size, and orientation of electronegative β substituents.

Since these protected diazepinones are novel, the ACD/I-Lab ¹H NMR Predictor, a service provided Advanced Chemistry Development, Inc. (ACD/Labs at ilab.acdlabs.com), was used to estimate splitting pattern and chemical shifts of an dibenzyl 6,7-dihyroimidazo[4,5-d][1,3]diazepin-8-(3H)-one (FIGS. 6I and 6J) to further confirm the appearance of the methylene (—CH₂—) functional group. FIG. 6I shows a 400 MHz ACD/HNMR spectrum of dibenzyl 6,7-dihyroimidazo[4,5-d][1,3]diazepin-8-(3H)-one in non-polar and non-aromatic solvent. FIG. 6J shows an expanded view of a 400 MHz ACD/HNMR spectrum of dibenzyl 6,7-dihyroimidazo[4,5-d][1,3]diazepin-8-(3H)-one between 4.30 to 3.90 ppm, where the cyclic —CH₂— group appears, in non-polar and non-aromatic solvent.

The program predicted the methylene (—CH₂—) hydrogen nuclei to be diastereomeric, contrary to the observed data (FIG. 6D and literature (Chan, E.; Putt, S. R.; Showalter, H. D. H.; Baker, D. C. J. Org. Chem. 1982, 47, 3457-3464)); but it was simulated in a non-polar and non-aromatic solvent and may not be entirely accurate. However, the chemical shifts for the diastereomeric hydrogen nuclei (4.25 and 3.98 ppm) were only about 0.1 ppm off from the observed methylene singlets of the three dibenzyl 6,7-dihyroimidazo[4,5-d][1,3]diazepin-8-(3H)-one isomers (4.12, 4.01, and 3.92 ppm). Thus, the cyclic —CH₂— nuclei of these three isomers appear to be similar and have HCH angles close to 120°.

There are numerous procedures in the literature for removal of the benzyl protective group, which include, but are not limited to: palladium-charcoal catalyzed hydrogenation with formic acid/methanol; 20% Pd(OH)₂/ethanol; Na, NH₃; hv, 405 nm (CuSO4: NH₃); CCl₃CH₂OCOCl/acetonitrile; and RuO₄/NH₃/water. The most common is palladium-charcoal catalyzed hydrogenation with H₂ gas. For improved scalability, formamide could be used instead of H₂ gas.

As an example, dibenzyl 6,7-dihyroimidazo[4,5-d][1,3]diazepin-8-(3H)-one (2 mmol) is suspended in methanol (50 mL), tetrahydrofuran (25 mL) and formic acid (1.0 mL). Palladium (5%)-charcoal was added (200 mg) before the mixture is rigorously stirred under 10-30 atm of H₂ at 50° C. until complete reaction (24 to 48 hours), as indicated by TLC (1:1:0.2 petroleum ether-ethyl acetate-methanol). The catalyst is filtered over Celite and thoroughly washed with methanol. Evaporation of the filtrate leaves solid containing isomeric mixture of diazepinol 8b.

Under dry condition, a mixture of diazepinol 8b and N,N-bis(trimethylsilyl) trifluoroacetamide (6.0 mmol), pyridine (6.0 mmol), and anhydrous acetonitrile (5 mL) is stirred for not less than 12 hours or until complete reaction. Excess reagents and solvents are evaporated at 60° C. More acetonitrile (20 mL) is added, stirred to homogeneity, and evaporated at 60° C. to give a silylated intermediate. It is re-suspended in anhydrous acetonitrile (15 mL) and cooled to −35 to −50° C. before anhydrous tin(IV) chloride (4 mmol) is added. About ten minutes later 2-deoxy-3,5-di-O-p-chlorobenzoyl-D-pentofuranosyl chloride in dry 1,2-dichloroethane is also added. The mixture is stirred for about one hour until complete reaction, as indicated by TLC (9:1 ethyl acetate-methanol). The solution is poured onto saturated bicarbonate solution (50 mL), diluted with ethyl acetate (50 mL) and filtered through Celite before the layers are separated and the aqueous layer extracted twice (2×50 mL) with ethyl acetate. The organic layers are combined, dried with magnesium sulfate and concentrated to dryness. The desired (8R)- and (8S)-3-(2-deoxy-3,5-di-O-p-benzyoyl-β-D-erythro-pentofuranosyl)-3,6,7,8-tetrahydroimidazo[4,5-d][1,3]diazepin-8-ol are separated from the α-anomers by flash chromatography (95:5 ethyl acetate-methanol) before they are suspended in a solution of ammonia (greater than 5 fold excess) in methanol (200 mL) and the mixture is stirred at ambient temperature for 24 hours. Excess ammonia is removed, and if necessary, the methanolic solution is decolorized with activated carbon (200 mg) before evaporation to dryness. This diastereoisomeric mixture is separated by fractional crystallization in water-methanol and/or by a C-18 reverse-phase column (93:7 water-methanol) to give pure pentostatin.

2. Synthesis of Pentostatin from N,N-di-Boc Hypoxanthine

In this example, pentostatin is synthesized through the route of ring-expansion of N,N-di-Boc-protected hypoxanthines to generate diazepinone derivatives as outlined in FIG. 6A. FIG. 7 shows the deprotection of N,N-di-Boc diazepinones to diazepinone 8a and diazepinol 8b. Diazomethane was prepared as described in Example 1 above.

N,N-Di-Boc hypoxanthine (531 mg, 1.58 mmol) was dissolved in anhydrous dichloromethane (50 mL) in a 250 mL round bottom flask before boron trifluoride diethyl etherate (0.2 mL, 1.3 mmol) was added. The mixture was closed with a rubber septum under N₂-atmosphere and cooled in an ice bath before the above freshly prepared diazomethane etherate was slowly added via a syringe to prevent over bubbling and addition of too much diazomethane. The mixture was stirred in the ice bath for about 30 minutes under N₂-atmosphere for the reaction to be completed, as indicated by TLC (1:1:0.2 petroleum ether-ethyl acetate-methanol), at which point the product could be seen precipitating. Anhydrous diethyl ether or hexane (200 mL) was added, the flask was flushed with N₂-gas, closed with a rubber septum and stored in the freezer (0° C.) overnight (12 hours). The white solid N,N-di-tert-butoxycarbonyl 6,7-dihyroimidazo[4,5-d][1,3]diazepin-8-(3H)-one was filtered, rinsed with diethyl ether (50 mL) and dried in vacuo for at least 6 hours.

There are numerous procedures in the literature for removal of the Boc protective group, which include, but are not limited to: Acetyl chloride/methanol; CF₃CO₂H/PhSH; TsOH/THF/CH₂Cl₂; 10% H₂SO₄/dioxane; Me₃SiI/acetonitrile; Me₃SiCl/phenol/CH₂Cl₂; SiCl₄/phenol/CH₂Cl₂; TMSOTf/PhSCH₃; Me₃SO₃H/dioxane/CH₂Cl₂; CF₃CO₂H/CH₂Cl₂; BF₃-Et₂O/4A ms/CH₂Cl₂/23° C./20 h; SnCl₄/AcOH/THF/CH₂Cl₂/toluene or acetonitrile; and ZnBr₂/CH₂Cl₂. Acetyl chloride in methanol generates anhydrous HCl in methanol. This is a convenient method for removing the Boc protection to give 8a.

Under dry condition, a mixture of diazepinone 8a, N,N-bis(trimethylsilyl) trifluoroacetamide (6.0 mmol), pyridine (6.0 mmol), and anhydrous acetonitrile (5 mL) is stirred for not less than 12 hours or until complete reaction. Excess reagents and solvents are evaporated at 60° C. More acetonitrile (20 mL) is added, stirred to homogeneity, and evaporated at 60° C. to give a silylated intermediate. It is re-suspended in anhydrous acetonitrile (15 mL) and cooled to −35 to −50° C. before anhydrous tin(IV) chloride (4 mmol) is added. About ten minutes later 2-deoxy-3,5-di-O-p-chlorobenzoyl-D-pentofuranosyl chloride in dry 1,2-dichloroethane is also added. The mixture is stirred for about one hour until complete reaction, as indicated by TLC (9:1 ethyl acetate-methanol). The solution is poured onto saturated bicarbonate solution (50 mL), diluted with ethyl acetate (50 mL) and filtered through Celite before the layers are separated and the aqueous layer extracted twice (2×50 mL) with ethyl acetate. The organic layers are combined, dried with magnesium sulfate and concentrated to dryness. The desired 3-(2-deoxy-3,5-di-O-p-benzyoyl-β-D-erythro-pentofuranosyl)-3,6,7,8-tetrahydroimidazo[4,5-d][1,3]diazepin-8 (3H)-one is separated from the α-anomer by flash chromatography (95:5 ethyl acetate-methanol) before they are suspended in a solution of ammonia (greater than 5 fold excess) in methanol (200 mL) and the mixture is stirred at ambient temperature for 24 hours. Excess ammonia is removed and solvent evaporated at 60° C. to give 9c. The crude intermediate is dissolved in water (10 mL) and methanol (10 mL) before sodium borohydride (1 mmol) was added. The solution is stirred at ambient temperature for one hour, at the end of which excess borohydride is decomposed by addition of dry ice. Methanol is removed by evaporation, and the aqueous solution is decolorized with activated carbon (200 mg) and filtered before lyophilization to a fluffy solid. This diastereoisomeric mixture is separated by fractional crystallization in water-methanol and/or by a C-18 reverse-phase column (93:7 water-methanol) to give pure pentostatin.

3. Syntheis of pentostatin from 2′-deoxyinosine

In this example, pentostatin is synthesized via the route of direct ring-expansion of 2′-deoxyinosine as outlined in FIG. 13. Diazomethane is prepared as described in Example 1.

Under dry condition, a mixture of 2′-deoxyinosine (2.0 mmol) and N,N-bis(trimethylsilyl) trifluoroacetamide (6.0 mmol), pyridine (6.0 mmol), and anhydrous acetonitrile (5 mL) is stirred for not less than 12 hours or until complete reaction. Excess reagents and solvents are evaporated at 60° C. More acetonitrile (20 mL) is added, stirred to homogeneity, and evaporated at 60° C. to give per-O-silylated 2′-deoxyinosine. The starting material is dissolved in anhydrous dichloromethane (50 mL) in a 250 mL round bottom flask before boron trifluoride diethyl etherate (0.2 mL, 1.3 mmol) is added. The mixture is closed with a rubber septum under N₂-atmosphere and cooled in an ice bath before the above freshly prepared diazomethane etherate is slowly added via a syringe to prevent over bubbling and addition of too much diazomethane. The mixture is stirred in the ice bath for about 30 minutes under N₂-atmosphere for the reaction to be completed, as indicated by TLC. A solution of tetrabutylammonium fluoride (3.0 mmol) dissolved in tetrahydrofuran (100 mL) is slowly added, and the mixture is stirred in the ice bath for not less than two hours, at which point intermediate product 9c can be seen precipitating. The crude intermediate is filtered, dried in vacuo, and then dissolved in water (10 mL) and methanol (10 mL) before sodium borohydride (1 mmol) is added. The solution is stirred at ambient temperature for one hour, at the end of which excess borohydride is decomposed by addition of dry ice. Methanol is removed by evaporation, and the aqueous solution is decolorized with activated carbon (200 mg) and filtered before lyophilization to a fluffy solid. This diastereoisomeric mixture is separated by fractional crystallization in water-methanol and/or by a C-18 reverse-phase column (93:7 water-methanol) to give pure pentostatin.

1. A method for preparing pentostatin, comprising:
providing a hypoxanthine derivative wherein at least one of the imidazole secondary amine and the O—C—N functionality (O═C—NH or HO—C═N) is protected by a protective group;
expanding the 6-member ring of the hypoxanthine derivative to form a protected diazepinone precursor having the formula 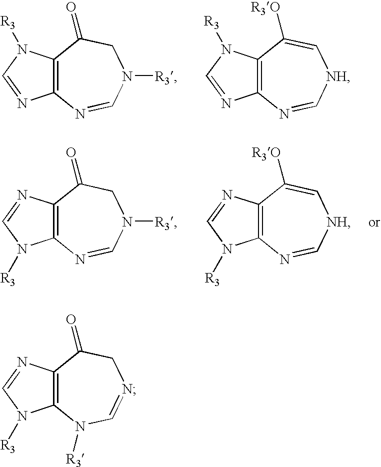
deprotecting the protected diazepinone precursor to yield a diazepinone precursor 8a having the formula 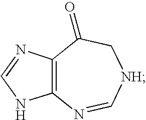
condensing the N-2 of the diazepinone precursor 8a with the C-1 of 2-deoxy-D-ribose or its derivative to yield an intermediate having the formula 9a 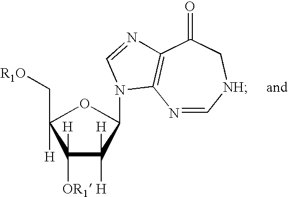
reducing the 8-keto functional group of the compound 9a to yield pentostatin,
wherein R₁ and R₁′ are each independently H or a protective group, and R₃ and R₃′ are each independently H or a protective group.

2. The method according to claim 1, wherein R₃ and R₃′ are each independently a carbamate, amide, aryl amine or silyl amine protective group.

3. The method according to claim 2, wherein the carbamate protective group is selected from the group consisting of methyl, ethyl, t-butyl, benzyl, 9-fluorenylmethyl, 2,2,2-trichloroethyl, 1-methyl-1-(4-biphenyl)ethyl, and 1-(3,5-di-t-butyl)-1-methylethyl.

4. The method according to claim 2, wherein the amide protective group is selected from the group consisting of acetamide, trifluoroacetamide, and benzamide.

5. The method according to claim 2, wherein the aryl amine protective group is selected from the group consisting of benzylamine, 4-methoxybenzylamine, and 2-hydroxybenzylamine.

6. The method according to claim 1, wherein R₁ and R₁′ are each independently a protective group selected from the group consisting of ether, ester, carbonate, sulfonate, cyclic acetal and ketal, chiral ketone, cyclic ortho ester, silyl derivative, cyclic carbonate, and cyclic borate.

7. The method according to claim 1, wherein R₁ and R₁′ are each independently a protective group of p-toluoyl.

8. The method according to claim 6, wherein the ether protective group is selected from the group consisting of methoxymethyl, benzyloxymethyl, allyl, propargyl, p-chlorophenyl, p-methoxypehenyl, p-nitrophenyl, benzyl, p-methoxybenzyl, dimethoxybenzyls, nitrobenzyl, halogenated benzyls, cyanobenzyls, trimethylsilyl, trimethylsilyl, triisopropylsilyl, tribenzylsilyl, and alkoxysilyls.

9. The method according to claim 6, wherein the ester protective group is selected from the group consisting of acetates and benzoates.

10. The method according to claim 6, wherein the carbonate protective group is selected from the group consisting of methoxymethyl, 9-fluorenylmethyl, 2,2,2-trichloroethyls, vinyl, allyl, nitrophenyls, and benzyls.

11. The method according to claim 6, wherein the sulfonate protective group is selected from the group consisting of allylsulfonate, mesylate, benzylsulfonate, and tosylate.

12. The method according to claim 6, wherein the cyclic acetal or ketal protective group is selected from the group consisting of methylene, ethylidene, acrolein, isopropylidene, cyclopentylidene, cyclohexylidene, cycloheptylidene, benzylidenes, mesitylene, 1-naphthaldehyde acetal, benzophenone ketal, and o-xylyl ether.

13. The method according to claim 6, wherein the chiral ketone protective group is selected from the group consisting of camphor and menthone.

14. The method according to claim 6, wherein the cyclic ortho ester protective group is selected from the group consisting of methoxymethylene, ethoxymethylene, 1-methoxyethylidene, methylidene, phthalide, ethylidene and benzylidene derivatives, butane-2,3-bisacetal, cyclohexane-1,2-diacetal, and dispiroketals.

15. The method according to claim 6, wherein the silyl derivative protective group is selected from the group consisting of di-t-butylsilylene and dialkylsilylene groups.

16. The method according to claim 1, wherein expanding the 6-member ring of the hypoxanthine derivative includes reacting the hypoxanthine derivative with diazomethane or trimethylsilyldiazomethane in the presence of a Lewis acid catalyst.

17. The method according to claim 16, wherein the Lewis acid catalyst is selected from the group consisting of trimethylsilyl triflate (TMSOTf), BX₃, AlX₃, FeX₃, GaX₃, SbX₅, SnX₄, AsX₅, ZnX₂, and HgX₂, wherein X is a halogen.

18. The method according to claim 16, wherein the Lewis acid catalyst is BF₃-Et₂O, ZnCl₂ or HgBr₂.

19. The method according to claim 16, wherein reacting the hypoxanthine derivative with diazomethane or trimethylsilyldiazomethane includes reacting the hypoxanthine derivative with anhydrous solution of diazomethane or trimethylsilyldiazomethane in ether.

20. A compound that is a precursor of pentostatin or other heterocyclic compounds having the formula 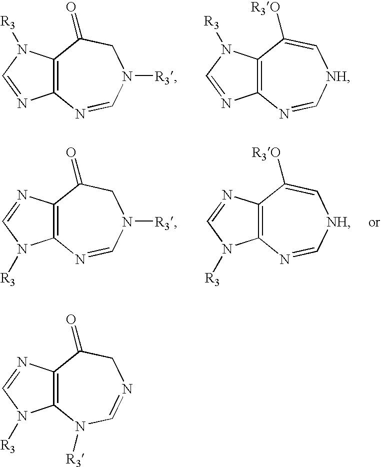
wherein R₃ and R₃′ are each independently H or a protective group, and at least one of R₃ and R₃′ is a protective group.

21. The compound according to claim 20, wherein R₃ and R₃′ are each independently a carbamate, amide, aryl amine or silyl amine protective group.

22. The compound according to claim 21, wherein the carbamate protective group is selected from the group consisting of methyl, ethyl, t-butyl, benzyl, 9-fluorenylmethyl, 2,2,2-trichloroethyl, 1-methyl-1-(4-biphenyl)ethyl, and 1-(3,5-di-t-butyl)-1-methylethyl.

23. The compound according to claim 21, wherein the amide protective group is selected from the group consisting of acetamide, trifluoroacetamide, and benzamide.

24. The compound according to claim 21, wherein the aryl amine protective group is selected from the group consisting of benzylamine, 4-methoxybenzylamine, and 2-hydroxybenzylamine.

25. A method for manufacturing a diazepinone precursor of pentostatin, comprising
protecting hypoxanthine at one or more locations using a protecting group;
reacting the protected hypoxanthine under a suitable condition in a appropriate solvent to yield a protected diazepinone precursor having the formula 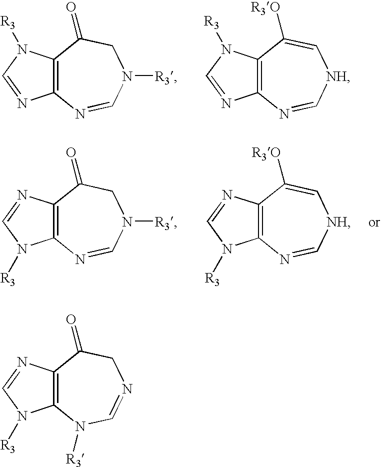
wherein R₃ and R₃′ are each independently H or a protective group;
precipitating the protected diazepinone precursor, and
deprotecting the protected diazepinone precursor to yield the diazepinone precursor 8a having the formula

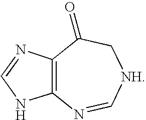

26. A method for manufacturing pentostatin, comprising:
protecting hypoxanthine at one or more locations using a protecting group;
reacting the protected hypoxanthine under a suitable condition in a appropriate solvent to yield a protected diazepinone precursor having the formula 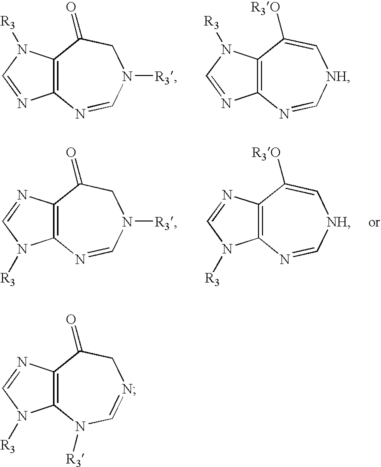
deprotecting the protected diazepinone precursor to yield the diazepinone precursor 8a having the formula 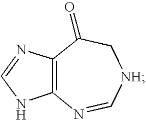
condensing the N-2 of the diazepinone precursor 8a with the C-1 position of 2-deoxy-D-ribose or its derivative to yield an intermediate 9a having the formula 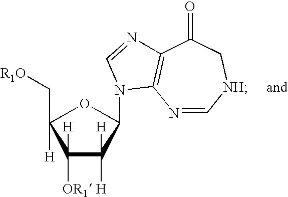
reducing the 8-keto functional group of the compound 9a to yield pentostatin,
wherein R₁ and R₁′ are each independently H or a protective group, and R₃ and R₃′ are each independently H or a protective group.

27. A method for preparing pentostatin, comprising:
providing a 2′-deoxyinosine derivative wherein at least one of the hypoxanthine oxygen, the hypoxanthine amide nitrogen, the 3′-hydroxyloxygen, and 5′-hydroxyloxygen is protected by a protective group;
expanding the O—C—N functionality (O═C—NH or HO—C═N) of the 6-member ring of the 2′-deoxyinosine derivative to produce an intermediate having the formula 20a or 20b 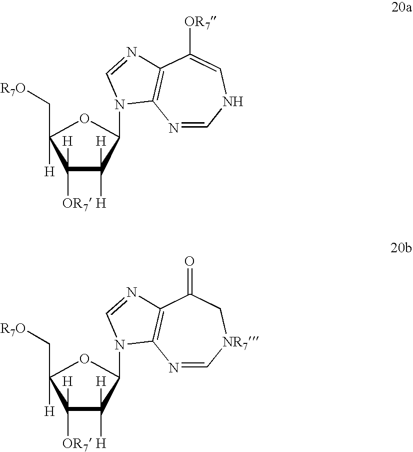
and
deprotecting and reducing the 8-keto functional group of compound 20a or 20b to yield pentostatin, wherein R₇, R₇′, R₇″ and R₇′″ are each independently H or a protective group.

28. The method according to claim 27, wherein R₇ and R₇′ are each independently a protective group selected from the group consisting of benzyl ethers, silyl ethers, and esters.

29. The method according to claim 28, wherein the benzyl ether protective group is selected from the group consisting of p-methoxybenzyl, 3,4-dimethoxybenzyl, nitrobenzyl, and p-cyanobenzyl.

30. The method according to claim 28, wherein the silyl ether protective group is selected from the group consisting of trialkylsilyl and alkoxydialkylsilyl.

31. The method according to claim 30, wherein the trialkylsilyl protective group is selected from the group consisting of trimethylsilyl, triethylsilyl, triisopropylsilyl, dimethylisopropylsilyl, diethylisopropylsilyl, t-butyldimethylsilyl, tribenzylsilyl, triphenylsilyl, di-t-butylmethylsilyl, and tris(trimethylsilyl)silyl.

32. The method according to claim 30, wherein the alkoxydialkylsilyl protective group is selected from the group consisting of t-butylmethoxyphenylsilyl and t-butoxydiphenylsilyl.

33. The method according to claim 28, wherein the ester protective group is selected from the group consisting of acetate, halogenatedacetate, alkoxyacetate, and benzoate.

34. The method according to claim 27, wherein R₇″ is a protective group is selected from the group consisting of benzyl ethers and silyl ethers.

35. The method according to claim 34, wherein the benzyl ether protective group is selected from the group consisting of p-methoxybenzyl, 3,4-dimethoxybenzyl, nitrobenzyl, and p-cyanobenzyl.

36. The method according to claim 34, wherein the silyl ether protective group is selected from the group consisting of trialkylsilyl and alkoxydialkylsilyl.

37. The method according to claim 36, wherein the trialkylsilyl protective group is selected from the group consisting of trimethylsilyl, triethylsilyl, triisopropylsilyl, dimethylisopropylsilyl, diethylisopropylsilyl, t-butyldimethylsilyl, tribenzylsilyl, triphenylsilyl, di-t-butylmethylsilyl, and tris(trimethylsilyl)silyl.

38. The method according to claim 36, wherein the alkoxydialkylsilyl protective group is selected from the group consisting of t-butylmethoxyphenylsilyl and t-butoxydiphenylsilyl.

39. The method according to claim 27, wherein R₇′″ is a carbamate protective group.

40. The method according to claim 39, wherein the carbamate protective group is selected from the group consisting of methyl carbamate, ethyl carbamate, t-butyl carbamate, benzyl carbamate, 9-fluorenylmethyl carbamate, 2,2,2-trichloroethyl carbamate, 1-methyl-1-(4-biphenyl)ethyl carbamate, and 1-(3,5-di-t-butyl)-1-methylethyl carbamate.

41. The method according to claim 27, wherein expanding the O—C—N functionality (O═C—NH or HO—C═N) of the 6-member ring of the 2′-deoxyinosine derivative includes reacting the 2′-deoxyinosine derivative with diazomethane or trimethylsilyldiazomethane in the presence of a Lewis acid catalyst.

42. The method according to claim 41, wherein the Lewis acid catalyst is selected from the group consisting of trimethylsilyl triflate (TMSOTf), BX₃, AlX₃, FeX₃, GaX₃, SbX₅, SnX₄, AsX₅, ZnX₂, and HgX₂, wherein X is a halogen.

43. The method according to claim 41, wherein the Lewis acid catalyst is BF₃-Et₂O, ZnCl₂ or HgBr₂.

44. The method according to claim 41, wherein reacting the 2′-deoxyinosine derivative with diazomethane or trimethylsilyldiazomethane includes reacting the 2′-deoxyinosine derivative with anhydrous solution of diazomethane or trimethylsilyldiazomethane in ether.

45. The method according to claim 27, wherein R₇, R₇′, R₇″ and R₇′″ are each independently a silyl ether protective group.

46. The method according to claim 27, wherein deprotecting and reducing the 8-keto functional group of compound 20a or 20b includes
deprotecting compound 20a or 20b to produce a pentostatin precursor having the following formula 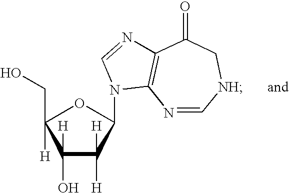
reducing the 8-keto functional group of the pentostatin precursor to yield pentostatin.

47. A method for preparing coformycin, comprising:
providing an inosine derivative wherein at least one of the hypoxanthine oxygen, the hypoxanthine amide nitrogen, the 2′-hydroxyloxygen, the 3′-hydroxyloxygen, and 5′-hydroxyl oxygen is protected by a protective group;
expanding the O—C—N functionality (O═C—NH or HO—C═N) of the 6-member ring of the 2′-inosine derivative to produce an intermediate having the formula 21a or 21b 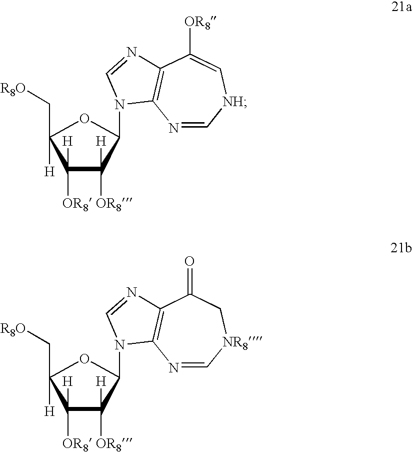
and
deprotecting and reducing the 8-keto functional group of compound 21a or 21b to yield coformycin, wherein R₈, R₈′, R₈″, R₈′″ and R₈″″ are each independently H or a protective group.

48. The method according to claim 47, wherein R₈, R₈′ and R₈′″ are each independently a protective group selected from the group consisting of benzyl ethers, silyl ethers, and esters.

49. The method according to claim 48, wherein the benzyl ether protective group is selected from the group consisting of p-methoxybenzyl, 3,4-dimethoxybenzyl, nitrobenzyl, and p-cyanobenzyl.

50. The method according to claim 49, wherein the silyl ether protective group is selected from the group consisting of trialkylsilyl and alkoxydialkylsilyl.

51. The method according to claim 50, wherein the trialkylsilyl protective group is selected from the group consisting of trimethylsilyl, triethylsilyl, triisopropylsilyl, dimethylisopropylsilyl, diethylisopropylsilyl, t-butyldimethylsilyl, tribenzylsilyl, triphenylsilyl, di-t-butylmethylsilyl, and tris(trimethylsilyl)silyl.

52. The method according to claim 50, wherein the alkoxydialkylsilyl protective group is selected from the group consisting of t-butylmethoxyphenylsilyl and t-butoxydiphenylsilyl.

53. The method according to claim 48, wherein the ester protective group is selected from the group consisting of acetate, halogenatedacetate, alkoxyacetate, and benzoate.

54. The method according to claim 47, wherein R₈″ is a protective group is selected from the group consisting of benzyl ethers and silyl ethers.

55. The method according to claim 54, wherein the benzyl ether protective group is selected from the group consisting of p-methoxybenzyl, 3,4-dimethoxybenzyl, nitrobenzyl, and p-cyanobenzyl.

56. The method according to claim 54, wherein the silyl ether protective group is selected from the group consisting of trialkylsilyl and alkoxydialkylsilyl.

57. The method according to claim 56, wherein the trialkylsilyl protective group is selected from the group consisting of trimethylsilyl, triethylsilyl, triisopropylsilyl, dimethylisopropylsilyl, diethylisopropylsilyl, t-butyldimethylsilyl, tribenzylsilyl, triphenylsilyl, di-t-butylmethylsilyl, and tris(trimethylsilyl)silyl.

58. The method according to claim 56, wherein the alkoxydialkylsilyl protective group is selected from the group consisting of t-butylmethoxyphenylsilyl and t-butoxydiphenylsilyl.

59. The method according to claim 47, wherein R₈″″ is a carbamate protective group.

60. The method according to claim 59, wherein the carbamate protective group is selected from the group consisting of methyl carbamate, ethyl carbamate, t-butyl carbamate, benzyl carbamate, 9-fluorenylmethyl carbamate, 2,2,2-trichloroethyl carbamate, 1-methyl-1-(4-biphenyl)ethyl carbamate, and 1-(3,5-di-t-butyl)-1-methylethyl carbamate.

61. The method according to claim 47, wherein expanding the 6-member ring of the inosine derivative includes reacting the inosine derivative with diazomethane or trimethylsilyldiazomethane in the presence of a Lewis acid catalyst.

62. The method according to claim 61, wherein the Lewis acid catalyst is selected from the group consisting of trimethylsilyl triflate (TMSOTf), BX₃, AlX₃, FeX₃, GaX₃, SbX₅, SnX₄, AsX₅, ZnX₂, and HgX₂, wherein X is a halogen.

63. The method according to claim 61, wherein the Lewis acid catalyst is BF₃-Et₂O, ZnCl₂ or HgBr₂.

64. The method according to claim 61, wherein reacting the inosine derivative with diazomethane or trimethylsilyldiazomethane includes reacting the hypoxanthine derivative with anhydrous solution of diazomethane or trimethylsilyldiazomethane in ether.
